(19) United States
(12) Patent Application Publication
(10) Pub. No.: US 2024/0058275 A1
(43) Pub. Date: Feb. 22, 2024

(54) NANO-SILICA - IN YEAST PARTICLE (YP) DRUG ENCAPSULATION APPROACH FOR IMPROVED THERMAL AND HYDROLASE STABILITY OF YP DRUG DELIVERY FORMULATIONS

(71) Applicant: UNIVERSITY OF MASSACHUSETTS, Boston, MA (US)

(72) Inventors: Gary R. Ostroff, Worcester, MA (US); Ernesto R. Soto, Worcester, MA (US)

(21) Appl. No.: 18/226,832

(22) Filed: Jul. 27, 2023

Related U.S. Application Data
(60) Provisional application No. 63/393,490, filed on Jul. 29, 2022.

(51) Int. Cl. A61K 9/50; A61K 47/69; A61K 47/38; B82Y 5/00; C12N 1/16

(52) U.S. Cl. CPC A61K 9/5068; A61K 47/6935; A61K 47/38; B82Y 5/00; C12N 1/16

(57) ABSTRACT

The present disclosure provides an improved yeast particle encapsulated nano-silica delivery system. The disclosure further provides methods of making and methods of using a nano-silica yeast particle delivery system.

### RELATED APPLICATIONS

The present invention claims the benefit of U.S. Provisional Patent Application Ser. No. 63/393,490, filed Jul. 29, 2022, the content of which is incorporated herein by reference in its entirety for all purposes.

### FIELD OF THE INVENTION

The present invention relates to medicine, pharmacology, and agriculture. More specifically, the present invention relates to yeast particles comprising ensilicated payloads.

### BACKGROUND

Drug delivery systems are designed to provide a biocompatible reservoir of an active agent for the controlled release of the active agent dependent either on time, or on local conditions, such as pH. There has been continuing interest in microscopic drug delivery systems such as microcapsules, microparticles and liposomes.

Yeast particles (YPs) are hollow, spherical particles about 2-4 μm in diameter that can be used for delivery of a drug payload. Due to their beta-glucan content, yeast particles can be targeted to phagocytic cells, such as macrophages and cells of lymphoid tissue.

Previous efforts to encapsulate payloads inside YPs include loading of soluble payload and trapping polymer components through the glucan hydrocolloid shell, and reacting them to form insoluble complexes trapped inside the shells, or through the layer by layer (LbL) absorption of a soluble payload component(s) onto the surface of a pre-existing YP encapsulated polyplexes or preformed nanoparticles. These trapping methods offer the advantages of very efficient payload trapping, phagocytic cell-targeted uptake of the YPs, and payload release in macrophages and dendritic cells. YP encapsulating nanocomplexes composed of serum albumin-yeast RNA (yRNA), inorganic crystalline matrices such as insoluble calcium, alum hydrocolloids, nanoplexes (chitosan, calcium alginate) are amongst vaccine formulation methods that have been published as effective and biocompatible, and resulting in strong, protective immune responses in animal models after systemic administration. However, these current YP drug delivery formulations still have limitations as these methods do not provide for room temperature thermal stability to eliminate the formulation cold chain storage process, and do not offer significant protection against acid and hydrolase degradation following oral delivery through the stomach and small intestine.

Thus, there is a need in the pharmaceutical, consumer and agricultural arts for the development of compositions and methods for delivering larger amounts of payloads to cells and organisms. YP delivery systems that encapsulate increased amount of payload, are stable, and allow controlled release of payload are needed.

### SUMMARY

In a first aspect, a nano-silica yeast particle (YP) delivery system is provided comprising a YP with a hollow inner cavity and at least one first payload, wherein the at least one first payload is substantially encapsulated by a nano-silica cage, and wherein the nano-silica cage and the at least one first payload are both substantially encapsulated within the hollow inner cavity of the YP.

In another aspect, a nano-silica yeast particle (YP) delivery system is provided comprising a YP with a hollow inner cavity and at least one first payload, wherein the at least one first payload is completely encapsulated by a nano-silica cage, and wherein the nano-silica cage and the at least one first payload are both confined within the hollow inner cavity of the YP.

In certain exemplary embodiments, the YP is selected from the group consisting of a yeast cell wall particle (YCWP), a glucan particle (GP), a yeast glucan particle (YGP), a yeast glucan-mannan particle (YGMP), a glucan lipid particle (GLP), a whole glucan particle (WGP), a glucan mannan lipid particle (GMLP), and a glucan chitin particle (GCP) or any mixtures thereof.

In certain exemplary embodiments, the first payload is selected from the group consisting of a protein, a peptide, a peptide antigen, an enzyme, an antibody, an antigen binding fragment of an antibody, a single stranded nucleic acid, and a double stranded nucleic acid, or any mixtures thereof.

In certain exemplary embodiments, the nano-silica cage comprises a chemical selected from the group consisting of tetraethylorthosilicate (TEOS), tetraethylorthogermanate (TEOG), tetramethylorthosilicate (TMOS), aminopropyl triethoxysilicate (APTES), Bis[3-(triethoxysilyl)propyl]disulfide (BTEPDS), and 3-(triethoxysylyl)-propyllisocyanate (TEPI) or any combinations thereof.

In certain exemplary embodiments, the nano-silica cage comprises polymerized tetraethylorthosilicate (TEOS).

In certain exemplary embodiments, the nano-silica cage comprises polymerized tetraethylorthogermanate (TEOG).

In certain exemplary embodiments, the nano-silica in YP delivery system further comprises a coating polymer in the hollow inner cavity, wherein the coating polymer is located on the outside of the nano-silica cage, and wherein the coating polymer is nontoxic and has no pharmacologic activity.

In certain exemplary embodiments, the coating polymer resists breakdown in the presence of gastric fluids in the oral cavity, esophagus, or stomach.

In certain exemplary embodiments, the coating polymer disintegrates in the small intestine.

In certain exemplary embodiments, the polymer is chosen from the group consisting of methacrylic acid methyl methacrylate copolymer, and methacrylic acid ethyl acrylate copolymer, cellulose acetate phthalate (CAP), cellulose acetate trimellate (CAT), hydroxy propyl methyl cellulose phthalate (HPMCP), dydroxyl propyl methyl cellulose acetate succinate (HPMCAS), polyvinyl acetate (PVAP), methacrylic acid polymer, and any combination thereof.

In certain exemplary embodiments, the payload is stable after a short-term or a long-term exposure to high temperature.

In certain exemplary embodiments, the payload is stable after exposure to a temperature of 25° C., 45° C., or 95° C.

In certain exemplary embodiments, the payload is stable after exposure to the high temperature for about 30 minutes, about 2 hours, about 5 hours, 15 days, 30 days, 45 days, 60 days, 75 days or 90 days.

In certain exemplary embodiments, the nano-silica in YP delivery system further comprises a one or more additional payloads, optionally wherein the one or more additional payload is not confined in the nano-silica cage.

In certain exemplary embodiments, the one or more additional payload is selected from the group consisting of a protein, a peptide, a peptide antigen, an enzyme, an antibody, an antigen binding fragment of an antibody, a single stranded nucleic acid, a double stranded nucleic acid, and a mixture thereof.

In some exemplary embodiments, the nano-silica in YP delivery system further comprises a pharmaceutically acceptable carrier or excipient.

In certain exemplary embodiments, a kit is provided comprising a nano-silica in YP delivery system of any of the previous embodiments and optional instructions for use.

In a another aspect, a method for preparing a nano-silica in yeast particle (YP) delivery system is provided comprising the steps of: (a) loading a YP comprising a hollow inner cavity with at least one first payload; and (b) resuspending the YP in prepolymerized tetrahydroorthosilicate (TEOS) in half hydrodynamic volume, wherein the prepolymerized TEOS is prepolymerized at a pH of about 2 to about 4, wherein the TEOS polymerizes to form a nano-silica cage within the hollow inner cavity, and wherein the nano-silica cage substantially encapsulates the at least one first payload at an encapsulation efficiency of at least 90%, and wherein the nano-silica cage and the at least one first payload are both confined within the hollow inner cavity of the YP.

In another aspect, a method of preparing a nano-silica in yeast particle (YP) delivery system is provided comprising the steps of: (a) loading a YP comprising a hollow inner cavity with at least one first payload; and (b) resuspending the YP in prepolymerized tetrahydroorthosilicate (TEOS) in half hydrodynamic volume, wherein the prepolymerized TEOS is prepolymerized at a pH of about 2 to about 4, wherein the TEOS polymerizes to form a nano-silica cage within the hollow inner cavity, and wherein the nano-silica cage completely encapsulates the at least one first payload at an encapsulation efficiency of at least 90%, and wherein the nano-silica cage and the at least one first payload are both confined within the hollow inner cavity of the YP.

In some embodiments, the method of preparing a nano-silica in YP delivery system further comprises the step of loading one or more additional payloads in the YP.

In certain exemplary embodiments, the method of preparing a nano-silica in YP delivery system further comprises the step of loading a coating polymer in the YP.

In another aspect, a pharmaceutical composition is provided comprising a nano-silica yeast particle (YP) delivery system comprising a YP with a hollow inner cavity and at least one first payload, wherein the at least one first payload is substantially encapsulated by a nano-silica cage, and wherein the nano-silica cage and the at least one first payload are both confined within

In another aspect, a pharmaceutical composition is provided comprising a nano-silica yeast particle (YP) delivery system comprising a YP with a hollow inner cavity and at least one first payload, wherein the at least one first payload is completely encapsulated by a nano-silica cage, and wherein the nano-silica cage and the at least one first payload are both confined within the hollow inner cavity of the YP.

In certain exemplary embodiments, the first payload is selected from the group consisting of a protein, a peptide, a nucleic acid,

In another aspect, a method is provided for treating a disease condition in a subject, comprising administering the pharmaceutical composition of any one of the embodiments above to a subject in need thereof.

In yet another aspect, a vaccine is provided comprising a nano-silica yeast particle (YP) delivery system comprising a YP with a hollow inner cavity and at least one first payload, wherein the at least one first payload is substantially encapsulated by a nano-silica cage, and wherein the nano-silica cage and the at least one first payload are both confined within the hollow inner cavity of the YP.

In yet another aspect, a vaccine is provided comprising a nano-silica yeast particle (YP) delivery system comprising a YP with a hollow inner cavity and at least one first payload, wherein the at least one first payload is completely encapsulated by a nano-silica cage, and wherein the nano-silica cage and the at least one first payload are both confined within the hollow inner cavity of the YP.

In certain exemplary embodiments, the first payload of the vaccine is selected from the group consisting of a protein, a peptide, a glycoprotein, a lipoprotein, a toxoid, a polysaccharide, and a nucleic acid or any combinations thereof.

In another embodiment, a method is provided for preventing a disease condition in a subject, comprising administering the vaccine of any one of the embodiments above to the subject in need thereof.

### BRIEF DESCRIPTION OF THE DRAWINGS

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1 is a schematic diagram showing ensilication of payloads within hollow glucan particles (GPs).

FIG. 2 shows the ensilication efficiency of fluorescently labeled Cy3-siDNA under different conditions. Sample 1: pH 2, HCl, 1 h at room temperature; sample 2: pH 2, HCl, 1 h at room temperature, GP-siRNA buffered at pH 7.

FIG. 3A-FIG. 3G show the various steps of a procedure for ensilication of payloads within GPs according to exemplary embodiments. Initial tetraethylorthosilicate (TEOS) (top phase, d=0.93 g/mL) and aqueous (acid or base) solution (bottom phase) (FIG. 3A) when mixed formed a TEOS-aqueous emulsion composed of pre-polymerized TEOS far from gel point (FIG. 3B). Upon further incubation, the TEOS-aqueous solution formed pre-polymerized TEOS close to gel point (FIG. 3C) which upon further incubation formed silica gel after full TEOS polymerization (FIG. 3D). When the TEOS-aqueous solution close to gel point (FIG. 3C) was loaded (FIG. 3E) into GPs containing payloads (FIG. 3F), full polymerization of TEOS inside GPs lead to ensilication of payloads within GPs (FIG. 3G).

FIG. 4A-FIG. 4B show efficiency of TEOS pre-polymerized at pH 2 to ensilicate rhodamine labeled bovine serum albumin (rBSA) within fluorescein labeled particles (fGPs). FIG. 4A shows a bar graph of percent of rBSA trapped in fGPs using various TEOS:rBSA ratios compared conventional methods of trapping payloads using yRNA or Ca₃(PO₄)₂. FIG. 4B shows brightfield and fluorescent microscopy overlays of fGP rBSA [SiO₂]ₙ formulation (at 12.5 w:w TEOS:rBSA).

FIG. 5 shows brightfield and fluorescent microscopy overlays of GPs with or without rBSA and fluorescent silica shell (TEOS/fluorescein labeled APTES).

FIG. 6A-FIG. 6B show the phagocytosis of fGP-BSA samples by macrophages. BSA was encapsulated within GPs via ensilication (FIG. 6A) or conventional trapping methods using yRNA (FIG. 6B).

FIG. 7 shows the cytotoxicity of fGP BSA [SiO₂]ₙ to B6 macrophage cell line.

FIG. 8 are fluorescent micrographs showing the intracellular release of rhodamine labeled BSA ensilicated in GPs or trapped within GPs using yRNA at 3 hours and 24 hours after uptake of GPs by macrophages.

FIG. 9 graphically depicts the effect of various pH and pre-polymerization times on TEOS ensilication efficiency. The W:W ratio of TEOS:BSA was 12.5:1. TEOS was pre-polymerized for 1, 6, or 20 hours prior to adding mixture to GP-BSA.

FIG. 10 graphically depicts the ensilication efficiency using TEOS pre-polymerized at pH 2 or pH 7 and GP-BSA prepared in water or phosphate buffered saline (PBS) at pH 7. The details of experimental conditions A, B, C, D are listed in Table 1.

FIG. 11 graphically depicts the ensilication efficiency after loading of BSA in GPs in the presence or absence of 6 M urea.

FIG. 12 graphically depicts the ensilication efficiency after loading of BSA in GPs in the presence or absence of high concentrations of sodium chloride (NaCl).

FIG. 13 graphically depicts the effect of TEOS pre-polymerization in strong or weak acids and TEOS-GP-BSA incubation conditions on ensilication efficiency.

FIG. 14A-FIG. 14C show a schematic of ensilication of a first payload followed by encapsulation of a second payload within GPs (FIG. 14A), encapsulation efficiency of ensilicated BSA and chitosan within GPs (FIG. 14B), and brightfield and fluorescent micrographs showing location of rBSA and f-chitosan within GPs (FIG. 14C).

FIG. 15A-FIG. 15B graphically depict efficient ensilication of BSA with TEOS or tetraethylorthogermanate (TEOG) at pH 2 (FIG. 15A) and pH 10 (FIG. 15B).

FIG. 16 graphically depicts the percent of alginate bound to GP with only TEOS and GPs with cationic alginate or anionic poly-lysine polymers partially ensilicated with TEOS.

FIG. 17 graphically depicts the percent of chitosan bound to GPs with only TEOS and GPs with cationic alginate or anionic poly-lysine polymers partially ensilicated with TEOS.

FIG. 18A-FIG. 18C schematically depict a procedure to measure BSA after exposure of GP-BSA-N021n to simulated gastric fluid (SGF)/pepsin or simulated intestinal fluid (SIF)/pancreatin (FIG. 18A) and graphically depict the percent BSA recovered after exposure to PBS (control) or SGF/pepsin (FIG. 18B) or SIF/pancreatin (FIG. 18C).

FIG. 19 shows the schematic of a procedure for preparation of EUDRAGIT® coated ensilicated payload within GPs.

FIG. 20A-FIG. 20B show a flowchart summarizing the steps for extraction and quantification of BSA after incubation of GP containing EUDRAGIT® coated ensilicated BSA with SGF/pepsin followed by SIF/pancreatin (FIG. 20A), and a graph showing BSA remaining in GPs after enzymatic digestion over a period of one hour (FIG. 20B).

FIG. 21A-FIG. 21D show the quantity of encapsulated and unencapsulated BSA after heat treatment as quantified by fluorescence (FIG. 21A) or SDS-PAGE densitometry (FIG. 21B), and the encapsulated BSA recovered from GPs exposed to either 95° C. or 20° C. for one hour separated on a denaturing gel (FIG. 21C) or on a native gel (FIG. 21D).

FIG. 22A-FIG. 22F show the various steps of the production of silicate capped yeast particles (GPs) according to an exemplary embodiment. Initial TEOS (top phase, d=0.93 g/mL) and aqueous ammonium hydroxide (NH₄OH) solution (bottom phase) (FIG. 22A) when mixed formed a TEOS—NH₄OH aqueous emulsion composed of pre-polymerized TEOS far from gel point (FIG. 22B). TEOS—NH₄OH aqueous solution formed pre-polymerized TEOS close to gel point (FIG. 22C) which rapidly fully polymerized to form silica gel (FIG. 22D). When pre-polymerized TEOS close to gel point (FIG. 22C) was incubated with GPs containing payloads (FIG. 22E), very rapid (<10 minutes) polymerization of TEOS in basic NH₄OH prevented TEOS from diffusing inside GPs, and instead resulted in polymerization of TEOS on the GP surface, resulting in plug sealed or silicate capped YPs (FIG. 22F).

FIG. 23A-FIG. 23B show the percent of BSA trapped in GPs in the absence of TEOS (control), in the presence of TEOS (ensilication approach), and in the presence of TEOS and ammonium hydroxide (NH₄OH; plug seal approach) (FIG. 23A). (FIG. 23B) shows the fluorescent micrographs of GPs containing ensilicated BSA and plug sealed GP-BSA.

FIG. 24 graphically depicts the percent of BSA trapped in GPs via ensilication in the presence of 0.01 M HCl (control) or 0.1%, 1%, and 10% ammonium hydroxide (NH₄OH) as a polymerization catalyst.

FIG. 25 graphically depicts anti-chicken ovalbumin (OVA) IgG antibody titers in control unvaccinated mice and mice immunized with OVA antigen and mouse serum albumin encapsulated in GPs by trapping with yRNA or by ensilication.

FIG. 26 graphically depicts the survival rate after exposure to a lethal cryptococcal infection of unvaccinated mice and mice vaccinated by standard GP Cpd1 MSA/yRNA and GP Cpd1 TEOS vaccines.

FIG. 27A-FIG. 27B graphically depict the survival rate (FIG. 27A) after exposure to a lethal cryptococcal infection of unvaccinated mice, and mice vaccinated by standard GP Cda2 MSA/yRNA and GP Cda2 TEOS vaccines and cryptococcal colony forming units (CFU) on day 70 post initial exposure (FIG. 27B).

FIG. 28 graphically depicts the efficiency of ensilication of lysozyme within GPs at various TEOS:lysozyme weight ratios.

FIG. 29A-FIG. 29C schematically depict a procedure to measure lysozyme (FIG. 29A), and shows percent of lysozyme remaining in dry (FIG. 29B) or 0.9% saline (FIG. 29C) GP-lysozyme samples after prolonged exposure to room or high temperature according to exemplary embodiments. Free lysozyme was used as control.

FIG. 30 graphically depicts the percent lysozyme activity retained after exposure to high temperature in samples containing free lysozyme or GP with ensilicated lysozyme (either dry/lyophilized sample or sample suspended in 0.9% saline).

FIG. 31A-FIG. 31B graphically depict the percent lysozyme activity retained by free lysozyme, lysozyme encapsulated within GPs, lysozyme trapped in GPs with yRNA, and lysozyme ensilicated within GPs during short-term incubation at 45° C. Samples were stored either as dry, lyophilized powders (FIG. 31A) or liquid samples stored in PBS with 2 mM sodium azide (NaN₃) (FIG. 31B).

FIG. 32 graphically depicts the percent lysozyme activity retained by free lysozyme and lysozyme ensilicated within GPs during long-term incubation at 45° C.

FIG. 33A-FIG. 33B show the release kinetics of GP-IgG-(SiO₂)ₙ IgG in a B6 cell line. The GP-IgG-(SiO₂)ₙ diffused throughout the cell cytoplasm after phagocytosis (FIG. 33A) and was detected in intact form for 5 hours (FIG. 33B).

FIG. 34A-FIG. 34B show the efficiency with which hairpin peptide was ensilicated within GPs or GMLPs using various TEOS:peptide weight ratios (FIG. 34A); fluorescent micrograph of GMLP-Peptide-SiO₂ prepared with TEOS:peptide ratio of 31.1 (FIG. 34B); and percent peptide extracted from GPs using HCl or NaF (FIG. 34C).

FIG. 35 shows an SDS-PAGE of a GP pellet and supernatant after ensilication of a CDa2 peptide vaccine in GPs.

FIG. 36 graphically depicts the encapsulation efficiency of different RNAs in GPs using 10% 3-aminopropyltriethoxysilane (APTES) and 90% tetraethylorthosilicate (TEOS).

FIG. 37A-FIG. 37B show the ensilication efficiency of the 300-mer Cy3 RNA within GPs using HCl at pH2 and pH 4 (FIG. 37A); location of RNA within GPs (FIG. 37B); extraction efficiency of RNA from GPs with NaF (FIG. 37C); and RNA extracted from GPs (FIG. 37D).

FIG. 38 graphically depicts the ensilication efficiency of fluorescently labeled ssDNA under different conditions.

FIG. 39A-39D depict SDS-PAGE gels showing the proteins in the supernatant (unencapsulated) and GP pellet (encapsulated) fractions after GPs were loaded with lysozyme (FIG. 39A), ovalbumin (FIG. 39B), transferrin (FIG. 39C) or glucose oxidase (FIG. 39D).

FIG. 40A-40B depict fluorescence micrographs with and without an overlay of a brightfield micrograph showing mCherry protein fluorescence in 3T3-D1 cells following transfection with GP yRNA:25k PEI mCherry mRNA and an endosomal release excipient control (FIG. 40A), and GP ensilicated mCherry mRNA without endosomal release excipient (FIG. 40B).

### DETAILED DESCRIPTION

The present disclosure improves upon conventional encapsulation technologies by providing a yeast particle (YP) delivery system comprising an extracted yeast cell wall and an ensilicated payload.

Silica based trapping methods have been previously reported for many drug delivery applications. Mesoporous silica nanoparticles (MSN) formed by polymerization of tetraethylorthosilicate (TEOS) around a sacrificial micelle frame have been extensively used for drug encapsulation. MSNs have been previously used for encapsulation of the chemotherapeutic doxorubicin (DOX). However, the chemical synthetic conditions needed for the formation of MSNs cannot be generated inside YPs. To overcome this synthetic hurdle, DOX delivery was attempted by non-covalently liking MSNs to the outer surface of YPs functionalized with anionic polymers. Although this approach worked, its drawbacks included limited payload loading capacity and poor stability of the MSN-DOX bound on the outer surface of YPs.

Encapsulation of proteins in silica-based “cages” prepared from tetraethylorthosilicate (TEOS) has been previously described by Chen et al. (Chen, Y-C, Smith, T, Hicks, R. H, Doekhie, A, Koumanov, F, Wells, S. A, Edler, K. J, Van Den Eisen, J, Holman, G. D, Marchbank, K. J, Sartbaeva, A. Tailored stability, storage and release of proteins with tailored fit in silica, Nature Scientific Reports, 2017, 7:46568) and Wahid et al. (Wahid, A. A., Doekhie, A., Sartbaeva, A. et al. Ensilication Improves the Thermal Stability of the Tuberculosis Antigen Ag85b and an Sbi-Ag85b Vaccine Conjugate. Sci Rep 9, 11409 (2019)). The protein-silica complex provides protection against heat denaturing of protein.

The use of TEOS and other silicates on yeast particles has been reported for the preparation of vaccines comprising YPs loaded with the vaccine and a polymerized silicate contacting the YP such that the YP is capped by the silicate (plug seal approach) (Wagner, T. E, Vaccine Delivery Systems Using Yeast Cell Wall Particles, 2017, U.S. Pat. No. 10,166,195, and United States Patent Application publication 2017/0007688 A1).

In the “ensilication” approach, a prepolymerized TEOS is reacted with a payload leading to formation of a rigid silica shell surrounding the payload molecules, thus protecting the payload from external factors that could lead to denaturing and/or degradation to form an insoluble complex. The reported procedure was developed to produce large ensilicated protein particles (average diameter>700 nanometers). Such large particles cannot be loaded inside YPs.

The subject disclosure is based in part on the discovery that pre-polymerized TEOS could be absorbed by YPs to trap payloads via in situ ensilication inside the hollow cavity of YPs. Dry YPs loaded with payloads (e.g., nanoparticles, proteins, antibodies, nucleic acids, and the like) were incubated with prepolymerized TEOS or other silane compounds. Prepolymerized TEOS was then diffused into the hollow YPs and formed a silica shell around the payload molecules. The payloads were trapped with high efficiency inside the silica shell in the YP cavity. The YP carrying ensilicated payloads could be phagocytosed by macrophage cells. The payload was efficiently released inside phagocytic cells. In addition to retaining biological activity of payload while offering similar or higher encapsulation efficiency as previous methods, the instant YP ensilication method offers improved payload stability due to higher resistance to thermal or enzymatic degradation.

In the present disclosure, YPs carrying payload molecules are incubated with prepolymerized TEOS. Diffusion of TEOS into the YPs leads to ensilication of the payload inside the YPs such that the chemical or biologic activities of the payloads are not permanently altered or diminished. The compositions and methods of the present disclosure can yield a highly stable YP-payload delivery system with an increased resistance to thermal or enzymatic degradation, thereby, providing for a significant improvement over existing technologies.

The disclosures of patents, patent applications, and publications in their entireties are hereby incorporated by reference into this application in order to more fully describe the state of the art as known to those skilled therein as of the date of the invention described and claimed herein (e.g., U.S. Pat. Nos. 7,740,861, 8,389,485, 9,242,857, 9,655,360, 10,004,229, 10,166,195; European Patent No. 1711058, WO2005070213A2, WO2005113128A1 and associated patents/patent applications). The instant disclosure will govern in the instance that there is any inconsistency between the patents, patent applications, and publications and this disclosure.

That the disclosure may be more readily understood, select terms are defined below.

The term “about” in connection with numerical values and ranges means that the number comprehended is not limited to the exact number set forth herein, and is intended to refer to ranges substantially within the quoted range while not departing from the scope of the invention. As used herein, “about” will be understood by persons of ordinary skill in the art. For example, “about” means that ±10% of a particular numerical value following the term.

As used herein, the term “ensilication” or “ensilicated” means that a polymeric structure, like a “mesh net,” covers or coats the payload molecules such that payload molecules inside the YPs are retained or entrapped within a polymeric structure (“cage”) formed by a silicate.

As used herein, the term “capping” or “capped” means that a polymeric structure, like a “mesh net,” covers or coats the YPs such that the payload loaded within the YPs is retained or entrapped within. The polymeric structure can be formed by a silicate.

### Yeast Particles

As used herein, a “yeast particle” (YP) refers to readily available, biodegradable, substantially spherical, hollow particles of about 2-4 μm in diameter. YPs may be obtained as a byproduct of some food grade Baker's yeast (i.e., Saccharomyces cerevisiae) extract manufacturing processes. YPs include, but are not limited to, commercially available YPs (for example, Biongin® and SAFMANNAN®), extracted yeast cell wall particles (YCWPs), yeast cell particles (YCPs), glucan particles (GPs), yeast glucan particles (YGPs), yeast glucan-mannan particle (YGMP), glucan lipid particles (GLPs), whole glucan particles (WGPs), glucan mannan lipid particles (GMLPs) and the like. YPs comprise a intact ghost shell composed of remnants of cell wall components after yeast cell extraction and a large a hollow inner cavity. Methods of preparing extracted yeast cell wall particles are known in the art, and are described, for example in U.S. Pat. Nos. 4,992,540, 5,082,936, 5,028,703, 5,032,401, 5,322,841, 5,401,727, 5,504,079, 5,968,811, 6,444,448, 6,476,003, published U.S. applications 2003/0216346 A1, 2004/0014715 A1, 2021/0023017 A1, and PCT published application WO 02/12348 A2, which are specifically incorporated herein by reference.

### Hydration of Yeast Particles

A sufficient level of YP hydration is needed for encapsulation and release of payloads.

Dry YPs can be hydrated by incubation with a variety of aqueous solutions. Suitable aqueous solutions include, but are not limited to: water; saline, e.g., phosphate buffered saline; any buffer solution known in the art with a pH between 3 and 11; any acid solution known in the art with a pH≥1.5; any basic solution known in the art with a pH<11; any salt solution known in the art that does not chemically interfere with the payload, and the like.

### Payload Molecules

The nano-silica YPs of the present disclosure are useful for in vivo or in vitro delivery of payload molecules to a cell or an organism. Any molecular payload that can be ensilicated within the YP is envisioned by the present disclosure.

Payload can be a protein, a peptide, a nucleic acid, or a combination thereof.

A. Antimicrobial Payloads

Certain exemplary embodiments of the present disclosure provide for compositions and methods for the loading and delivery of payload molecules, such as antimicrobial peptides are effective against classes of organisms such as Gram-positive bacteria, Gram negative bacteria, fungi, and viruses.

A peptide with microbicidal or microbistatic inhibitory properties can be applied to an environment either presently exhibiting microbial growth (i.e., therapeutic treatment) or to an environment at risk of supporting such growth (i.e., prevention or prophylaxis). An environment capable of sustaining microbial growth refers to a fluid, substance, or organism where microbial growth can occur or where microbes can exist. Such environments can be, for example, animal tissue or bodily fluids, water and other liquids, food, food products or food extracts, crops, and certain inanimate objects. It is not necessary that the environment promote the growth of the microbe, only that it permit its subsistence.

The antimicrobial peptide component may comprise a single microbial or a mixture of antimicrobials.

Controlled release pharmaceutical dosage forms can be used to optimize drug delivery and enhance patient compliance. A pharmaceutical dosage form can deliver more than one drug, each at a modified rate.

B. Other Payloads

The YP delivery system of the present invention is useful for in vivo or in vitro delivery of payload molecules including, but limited to, single and double stranded natural and chemically modified polynucleotides such as oligonucleotides, antisense constructs, siRNA, enzymatic RNA, mRNA, and recombinant DNA constructs, including expression vectors.

In other exemplary embodiments, the YP delivery system of the present invention is useful for in vivo or in vitro delivery of payload molecules such as, peptides and proteins. By “protein” is meant a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, or at least about 20 kD.

Examples of proteins or peptides thereof encompassed within the definition herein include, but are not limited to: mammalian proteins, such as, e.g., growth hormone (GH), including human growth hormone, bovine growth hormone, and other members of the GH supergene family; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX tissue factor, and von Willebrand's factor; anti-clotting factors such as Protein C; atrial natriuretic factor; lung surfactant; a plasminogen activator, such as urokinase or tissue-type plasminogen activator (t-PA); bombazine; thrombin; alpha tumor necrosis factor, beta tumor necrosis factor; enkephalinase; RANTES (regulated on activation normally T-cell expressed and secreted); human macrophage inflammatory protein (MIP-1-alpha); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-beta; platelet-derived growth factor (PDGF); fibroblast growth factor such as aFGF and bFGF; epidermal growth factor (EGF); transforming growth factor (TGF) such as TGF-alpha and TGF-beta, including TGF-beta1, TGF-beta2, TGF-beta3, TGF-beta4, or TGF-beta5; insulin-like growth factor-I and -II (IGF-I and IGF-II); des(1-3)-IGF-I (brain IGF-D; insulin-like growth factor binding proteins; CD proteins such as CD3, CD4, CD8, CD19 and CD20; osteoinductive factors; immunotoxins; a bone morphogenetic protein (BMP); T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

Other examples of proteins or peptides thereof include members of the GH supergene family including, but not limited to, growth hormone, prolactin, placental lactogen, erythropoietin, thrombopoietin, interleukin-2, interleukin-3, interleukin-4, interleukin-5, interleukin-6, interleukin-7, interleukin-9, interleukin-10, interleukin-11, interleukin-12 (p35 subunit), interleukin-13, interleukin-15, oncostatin M, ciliary neurotrophic factor, leukemia inhibitory factor, alpha interferon, beta interferon, gamma interferon, omega interferon, tau interferon, granulocyte-colony stimulating factor, granulocyte-macrophage colony stimulating factor, macrophage colony stimulating factor, cardiotrophin-1 and other proteins identified and classified as members of the family.

In certain exemplary embodiments, a protein or peptide thereof includes one or more of the antigens described below.

The protein or peptide payload molecule is typically essentially pure and desirably essentially homogeneous (i.e., free from contaminating proteins etc.). “Essentially pure” protein means a composition comprising at least about 90% by weight of the protein, based on total weight of the composition, or at least about 95% by weight. “Essentially homogeneous” protein means a composition comprising at least about 99% by weight of protein, based on total weight of the composition. Proteins may be derived from naturally occurring sources or produced by recombinant technology. Proteins include protein variants produced by amino acid substitutions or by directed protein evolution (Kurtzman, A. L., et al., Advances in directed protein evolution by recursive genetic recombination: applications to therapeutic proteins, Curr Opin Biotechnol. 2001 12(4): 361-70) as well as derivatives, such as PEGylated proteins.

In certain embodiments, the protein is an antibody or an antigen-binding fragment thereof. The antibody or antigen-binding fragment thereof may bind to any of the above-mentioned molecules, for example. Exemplary molecular targets for antibodies encompassed by the present invention include: CD proteins such as CD3, CD4, CD8, CD19, CD20 and CD34; members of the HER receptor family such as the EGF receptor, HER2, HER3 or HER4 receptor; cell adhesion molecules such as LFA-1, Mol, p150,95, VLA-4, ICAM-1, VCAM and alphav/beta3 integrin including either alpha or beta subunits thereof (e.g. anti-CD11a, anti-CD18 or anti-CD11b antibodies); growth factors such as VEGF;

Other active agents that can be incorporated in the delivery system of the present invention include: gastrointestinal therapeutic agents such as aluminum hydroxide, calcium carbonate, magnesium carbonate, sodium carbonate and the like; digestants, enzymes and the like.

### Nano-Silica Cage

In certain embodiments, at least one payload of a YP is substantially encapsulated by a nano-silica cage. As used herein, a “nano-silica cage” refers to a covalently bonded silica matrix that can be used as described herein to surround a payload without covalently bonding with the payload to provide a physical barrier to release of the payload from the silica matrix. Optionally, the shape of the silica matrix closely matches the shape of the payload. In certain embodiments, a payload is “substantially encapsulated” by a nano-silica cage if it is about 50% to about 100% encapsulated. For example, the payload can be about 50%, about 55%, about 65%, about 70%, about 75%, about 80%, about 90%, about 91%, about 92%, about 93%, about 94%, about 95%, about 96%, about 97%, about 98%, about 99%, or about 100% encapsulated by a nano-silica cage.

### Payload Encapsulation Efficiency and Encapsulation Yield

Two parameters used to evaluate the payload encapsulation process are encapsulation yield (EY) and encapsulation efficiency (EE). EY, expressed as a percent value, is the weight ratio between payload and YP. EE, expressed as a percent value, is the weight ratio of the payload loaded or encapsulated in a YP with respect to the payload's initial mass. High EE is desirable to maximize payload delivery. In certain embodiments, EE can range from about 50% to about 100%. For example, the payload encapsulation efficiency can be about 50%, about 55%, about 65%, about 70%, about 75%, about 80%, about 90%, about 91%, about 92%, about 93%, about 94%, about 95%, about 96%, about 97%, about 98%, about 99%, or about 100%.

### Vaccines

In exemplary embodiments, the YP delivery system of the present invention is useful in providing oral delivery of vaccines. In exemplary embodiments, the system is used to deliver antigens, such as antigens of such microorganisms as Neisseria gonorrhea, Mycobacterium tuberculosis, Herpes virus (humonis, types 1 and 2), Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, equine herpes virus 1, equine arteritis virus, IBR-IBP virus, BVD-MB virus, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum and the like. In other embodiments, the system can be used to deliver neutralizing antibodies that counteract the above microorganisms.

In other embodiments, the system can be used to deliver enzymes such as ribonuclease, neuraminidase, trypsin, glycogen phosphorylase, sperm lactic dehydrogenase, sperm hyaluronidase, adenosinetriphosphatase, alkaline phosphatase, alkaline phosphatase esterase, amino peptidase, trypsin chymotrypsin, amylase, muramidase, acrosomal proteinase, diesterase, glutamic acid dehydrogenase, succinic acid dehydrogenase, beta-glycophosphatase, lipase, ATP-ase alpha-peptate gamma-glutamyl otransp epti das e, sterol-3-b eta-ol-dehydrogenase, DPN-diaphorase, glucocerebrosidase and other lysosomal hydrolases used for enzyme replacement therapies.

In exemplary embodiments, the system can deliver antigens of bioterrorism critical biological agents, including Category A agents such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain 0157:H7, Vibrio cholerae, Cryptosporidium parvum; and Category C agents such as nipah virus, hantaviruses, tickborne hemorrhagic fever viruses, tickborne encephalitis viruses, yellow fever, and multidrug-resistant tuberculosis.

In exemplary embodiments, the system can be used to deliver inactivated antigenic toxins, such as anatoxin antigens, including toxoids (inactivated but antigenic toxins), and toxoid conjugates. In exemplary embodiments, the toxoid is an inactivated microbial toxin. In other embodiments, the toxoid is an inactivated plant toxin. In further embodiments, the toxoid is an inactivated animal toxin. In certain embodiments, the system can be used to deliver toxoids such as pertussis toxoid, Corynebacterium diphtheriae toxoid, tetanus toxoid, Haemophilus influenzae type b-tetanus toxoid conjugate, Clostridium botulinum D toxoid, Clostridium botulinum E toxoid, toxoid produced from Toxin A of Clostridium difficile, Vibrio cholerae toxoid, Clostridium perfringens Types C and D toxoid, Clostridium chauvoei toxoid, Clostridium novyi (Type B) toxoid, Clostridium septicum toxoid, recombinant HIV tat IIIB toxoid, Staphylococcus toxoid, Actinobacillus pleuropneumoniae Apx I toxoid, Actinobacillus pleuropneumoniae Apx II toxoid, Actinobacillus pleuropneumoniae Apx III toxoid, Actinobacillus pleuropneumoniae outer membrane protein (OMP) toxoid, Pseudomonas aeruginosa elastase toxoid, snake venom toxoid, ricin toxoid, Mannheimia haemolytica toxoid, Pasteurella multocida toxoid, Salmonella typhimurium toxoid, Pasteurella multocida toxoid, and Bordetella bronchiseptica toxoid.

### Solvents, Loading Solvent, and Leave-In Solvents

Solvents may be added during the encapsulation process to facilitate loading of payloads in the YPs. Certain payloads of the present disclosure are water-insoluble or have low water solubility and may be loaded into YPs with a solvent that is compatible with yeast particles. In certain nonlimiting embodiments, the solvent may be an organic solvent. Suitable solvents include, but are not limited to, acetone, dichloromethane, ethyl acetate, alcohols such as ethanol or methanol, dimethylsulfoxide (DMSO), methanol-chloroform, hexane, petroleum ether, toluene, Neobee and the like. After a payload is completely encapsulated, the yeast particle and payloads may be processed to remove the solvent from the YP-payload formulation. Organic solvents such as acetone, dichloromethane, ethyl acetate, methanol, and DMSO may be unsafe for human administration and should be removed after a payload is completely encapsulated. Alternatively, the solvent used to facilitate payload encapsulation may be safe for human administration and can be left inside the YP along with the water-insoluble payload as a “leave-in solvent.”

### Surfactants

The term “surfactant,” as used herein, refers to any molecule having both a hydrophilic group (e.g., a polar group), which energetically prefers solvation by water, and a hydrophobic group which is not well solvated by water. The term “nonionic surfactant” is a known term in the art and generally refers to a surfactant molecule whose hydrophilic group (e.g., polar group) is not electrostatically charged.

Surfactants are generally low to moderate weight compounds which contain a hydrophobic portion, which is generally readily soluble in oil, but sparingly soluble or insoluble in water, and a hydrophilic portion, which is sparingly soluble or insoluble in oil, but readily soluble in water. In addition to protecting against growth and aggregation and stabilizing the organic compound delivery vehicle, surfactants are also useful as excipients in organic compound delivery systems and formulations because they increase the effective solubility of an otherwise poorly soluble or non-soluble organic compound, and may decrease hydrolytic degradation, decrease toxicity and generally improve bioavailability. Surfactants may also provide selected and advantageous effects on drug release rate and selectivity of drug uptake. Surfactants are generally classified as either anionic, cationic, zwitterionic, or nonionic.

Suitable surfactants include, but are not limited to, sodium lauryl sulphate, polysorbate 20, polysorbate 80, polysorbate 40, polysorbate 60, polyglyceryl ester, mono fatty acid ester of polyoxyethylene sorbitan, polyglyceryl monooleate, decaglyceryl monocaprylate, propylene glycol dicaprilate, poly ethylenepolypropylene glycol, triglycerol monostearate, polyoxyethylenesorbitan, monooleate, Tween®, Span® 20, Span® 40, Span® 60, Span® 80, IGEPAL®, Triton X-100, Neobee Brij 30 and the like, and any mixtures thereof.

### Temperature Stabilizing Agents

The storage stability of YPs containing payloads may be improved by addition of one or more temperature stabilizing agents. Common temperature stabilizing agents include sugars such sucrose, trehalose, glycerol, or sorbitol. Disaccharides such as sucrose and trehalose are natural cryoprotectants with good protective properties. A temperature stabilizing agent may comprise a carbohydrate component including between about 10% and 80% oligosaccharide, between about 5% and 30% disaccharide or between about 1% and 10% polysaccharide, and a protein component including between about 0.5% and 40% protein, e.g., hydrolyzed animal or plant proteins, based on the total weight of the composition. Ascorbic acid ions may be used in some embodiments for stabilization at higher temperature and humidity exposure. Alternatively, a combination of citrate and/or ascorbate ions with protein or protein hydrolysate may be used. In certain nonlimiting embodiments, the temperature stabilizing agent may be a glycerin. In certain nonlimiting embodiment temperature stabilizing agent may be glycerin at a concentration of about 5%, about 10%, about 15%, about 20%, about 25%, about 30%, about 35%, about 40%, about 45% or about 50%. In certain nonlimiting embodiment temperature stabilizing agent may be glycerin at a concentration of 1-5%, 5-10%, 10-15%, 15-20%, 20-25%, 25-30%, 35-40%, 40-45%, or 45%-50%.

### Enteric Coating Polymers

An enteric coating, also known as gastro-resistant coating is a barrier applied to oral medication that controls the location in the digestive tract where it is absorbed. The term “enteric” refers to the small intestine; therefore, enteric coatings resist breakdown of medication before it reaches the small intestine. Enteric coatings are employed when the drug substance is inactivated or destroyed in the acid secretion of the stomach or is particularly irritating to the gastric mucosa or when bypass of the stomach substantially enhances drug absorption. Modern enteric coatings are usually formulated with synthetic polymeric material often referred to as polyacids. These polymers contain ionizable functional groups that render them water-soluble at a specific pH value. Examples of coating polymers include cellulose acetate phthalate (CAP), cellulose acetate trimellate (CAT), hydroxy propyl methyl cellulose phthalate (HPMCP), dydroxyl propyl methyl cellulose acetate succinate (HPMCAS), polyvinyl acetate (PVAP), and methacrylic acid polymers. Several different types of Eudragit polymers with enteric release capabilities are commercially available in a wide range of different physical forms (aqueous dispersion, organic solution, granules and powders). The pH at which these polymers dissolve is dependent on the content of the carboxylic acid in the copolymer. Methacrylic acid methylmethacrylate copolymers (Eudragit L and S), and methacrylic acid ethyl acrylate copolymer (Eudragit L30D) are the exemplary choice of coating polymers for enteric formulations. They allow targeting of specific areas of the intestine.

### Articles of Manufacture, Compositions, and Methods

Certain embodiments of the present disclosure provide compositions and methods for use in controlling sucking and biting pests, including e.g., mosquitoes, ticks, lice, fleas, mites, flies, and spiders.

Certain embodiments of the present disclosure provide for compositions and methods for use in controlling nematodes. Nematodes (Kingdom: Animalia; Phylum: Nematoda) are microscopic round worms. They can generally be described as aquatic, triploblastic, unsegmented, bilaterally symmetrical roundworms, that are colorless, transparent, usually bisexual, and worm-shaped (vermiform), although some can become swollen (pyroform).

Many nematodes are obligate parasites and a number of species constitute a significant problem in agriculture. Thus, methods to control their parasitic activities are an important feature in maximizing crop production in modern intensive agriculture.

Nematodes are not just parasitic to plants but a number of species are parasitic to animals, both vertebrate and invertebrate. Around 50 species attack humans and these include Hookworm (Anclyostoma), Strongylids (Strongylus), Pinworm (Enterolobius), Trichinosis (Trichina), Elephantitis (Wuchereria), Heartworm (Dirofilaria), and Ascarids (Ascaris).

In some embodiments of the present disclosure, any of the compositions described above may be formulated in a deliverable form suited to a particular application. Deliverable forms that can be used in accordance with embodiments of the present disclosure include, but are not limited to, liquids, emulsions, emulsifiable concentrates, solids, aqueous suspensions, oily dispersions, pastes, granules, powders, dusts, fumigants, and aerosol sprays. Suitable deliverable forms can be selected and formulated by those skilled in the art using methods currently known in the art. The compositions can be provided in combination with an agriculturally, food, or pharmaceutically acceptable carrier or excipient in a liquid, solid, or gel-like form. For solid compositions, suitable carriers include pharmaceutical or food grades of mannitol, lactose, starch, magnesium stearate, sodium saccharin, talc, cellulose, glucose, sucrose, and magnesium carbonate. Suitably, the formulation is in tablet or pellet form. As suitable carrier could also be a human or animal food material. Additionally, conventional agricultural carriers could also be used.

The composition of the present disclosure may alternatively be applied via irrigation. This is suitable for treating nematodes or other soil borne pathogens or parasites.

In certain embodiments, the present disclosure provides for compositions in the form of granules and methods of controlling pests using the same. Granules allow for the use of less selective herbicides, pesticides, and combinations thereof, and thus offer a means to control pests that are not otherwise easily controlled. Granules are a convenient application form for producers with small allotments such as paddy rice farmers, or for growers of turf where spays are complicated by the needs of near neighbors sensitive to drift or odor or for broad acre farmers who wish to apply fertilizers and herbicides together and who do not have convenient access to water.

The granules may be used in flooded paddies, recently irrigated turf, or in areas where it is inconvenient or impossible to remove irrigation water. The granules allow small holders the means to apply crop protection chemicals without expensive equipment, and without risk of exposing airways or eyes to aerosols or spray materials. Granules can be easily measured and distributed by hand. Using granules that are designed for uniform dispersal is advantageous because it ensures even application, prevents post-harvest decay, and allows coating of seeds.

### Pharmaceutical Compositions and Administration

In addition, the compositions and methods of the present disclosure are useful in the fields of industrial and consumer products and medicines, e.g., in food, human and animal drugs, and cosmetics, and the like. In some embodiments, the disclosure provides for compositions and methods for use in both human and veterinary medicine. In certain embodiments, the present disclosure relates to therapeutic treatment of mammals, birds, and fish. For example, the compositions and methods of the present disclosure are useful for therapeutic treatment of mammalian species including, but not limited to, human, bovine, ovine, porcine, equine, canine, and feline species.

Routes of administration of the delivery system include but are not limited to oral, buccal, sublingual, pulmonary, transdermal, transmucosal, as well as subcutaneous, intraperitoneal, intravenous, and intramuscular injection. Exemplary routes of administration are oral, buccal, sublingual, pulmonary, and transmucosal.

The YP delivery system of the present disclosure is administered to a patient in a therapeutically effective amount. The YPs can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using a controlled release formulation. It is also noted that the dose of the compound can be varied over time. The YP delivery system can be administered using an immediate release formulation, or using a controlled release formulation, or combinations thereof. The term “controlled release” includes sustained release, delayed release, and combinations thereof, as well as release mediated by chemical (e.g., pH) and/or biological (e.g., enzyme) hydrolysis.

A pharmaceutical composition of the disclosure can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the animal or human treated, and further depending upon the route by which the composition is to be administered. By way of example, the composition can comprise between 0.1% and 100% (w/w) active ingredient. For example, the active ingredient weight in the pharmaceutical composition may be at least about 1%, 5%, 10%, 15%, 20%, 25%, 30%, 35%, 40%, 45%, 50%, 55%, 60%, 65%, 70%, 75%, 80%, 85%, 90%, 95%, or 100%. A unit dose of a pharmaceutical composition of the disclosure will generally comprise from about 100 milligrams to about 2 grams of the active ingredient, or from about 200 milligrams to about 1.0 gram of the active ingredient.

In addition, YP delivery system of the present disclosure can be administered alone, in combination with YPs with a different payload, or with other pharmaceutically active compounds. The other pharmaceutically active compounds can be selected to treat the same condition as the YPs with ensilicated payloads or a different condition.

If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup.

Another aspect of the disclosure relates to a kit comprising a pharmaceutical composition of the disclosure and instructional material. Instructional material includes a publication, a recording, a diagram, or any other medium of expression which is used to communicate the usefulness of the pharmaceutical composition of the disclosure for one of the purposes set forth herein in a human. The instructional material can also, for example, describe an appropriate dose of the pharmaceutical composition of the disclosure. The instructional material of the kit of the disclosure can, for example, be affixed to a container which contains a pharmaceutical composition of the disclosure or be shipped together with a container which contains the pharmaceutical composition. Alternatively, the instructional material can be shipped separately from the container with the intention that the instructional material and the pharmaceutical composition be used cooperatively by the recipient.

The disclosure also includes a kit comprising a pharmaceutical composition of the disclosure and a delivery device for delivering the composition to a human. By way of example, the delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit can further comprise an instructional material as described herein.

For example, a kit may comprise two separate pharmaceutical compositions comprising respectively a first composition comprising a particulate delivery system and a pharmaceutically acceptable carrier; and composition comprising second pharmaceutically active compound and a pharmaceutically acceptable carrier. The kit also comprises a container for the separate compositions, such as a divided bottle or a divided foil packet. Additional examples of containers include, without limitation, syringes, boxes, and bags. Typically, a kit comprises directions for the administration of the separate components. The kit form is advantageous when the separate components are administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms, e.g., tablets and capsules. Blister packs generally consist of a sheet of relatively stiff material covered with a foil of, e.g., a transparent plastic material. During the packaging process, recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “first week, Monday, Tuesday, . . . etc. . . . second week, Monday, Tuesday,” etc. Other variations of memory aids will be readily apparent.

Dosing can be hourly, e.g., every hour, every two hours, every four hours, every eight hours etc. Dosing can be weekly, biweekly, every four weeks, etc. A “daily dose” can be a single tablet or capsule or several pills or capsules to be taken on a given day. Also, a daily dose of a particulate delivery system composition can consist of one tablet or capsule, while a daily dose of the second compound can consist of several tablets or capsules and vice versa. The memory aid should reflect this and assist in correct administration.

In another embodiment of the present disclosure, a dispenser designed to dispense the daily doses one at a time in the order of their intended use is provided. The dispenser may be equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

A YP delivery system composition, optionally comprising other pharmaceutically active compounds, can be administered to a patient either orally, rectally, parenterally, (for example, intravenously, intramuscularly, or subcutaneously) intracisternally, intravaginally, intraperitoneally, intravesically, locally (for example, powders, ointments or drops), or as a buccal or nasal spray.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound. Parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the active ingredient combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols, e.g., propylene glycol, polyethylene glycol, and glycerol, and suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (e.g., powder or granular) form for reconstitution with a suitable vehicle (e.g., sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using anon-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, and sorbic acid. It may also be desirable to include isotonic agents, for example, sugars, and sodium chloride. Prolonged absorption of injectable pharmaceutical compositions can be brought about by the use of agents capable of delaying absorption, for example, aluminum monostearate and/or gelatin.

Dosage forms can include solid or injectable implants or depots. In certain embodiments, the implant comprises an aliquot of the particulate delivery system and a biodegradable polymer. In certain embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester), and a polyphosphazene.

Solid dosage forms for oral administration include capsules, tablets, powders, and granules. In such solid dosage forms, the particulate delivery system is optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the particulate delivery system can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or they can be coated using known methods to achieve delayed disintegration in the gastrointestinal tract of a human, thereby providing sustained release and absorption of the particulate delivery system, e.g. in the region of the Peyer's patches in the small intestine. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the particulate delivery system in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols. Hard capsules comprising the particulate delivery system can be made using a physiologically degradable composition, such as gelatin. Such hard capsules comprise the particulate delivery system, and can further comprise additional ingredients including, for example, an inert solid diluent such as calcium carbonate, calcium phosphate, or kaolin. Soft gelatin capsules comprising the particulate delivery system can be made using a physiologically degradable composition, such as gelatin. Such soft capsules comprise the particulate delivery system, which can be mixed with water or an oil medium such as peanut oil, liquid paraffin, or olive oil.

Oral compositions can be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the particulate delivery system to a specific location within the gastrointestinal tract. Such systems permit delivery at a predetermined time and can be used to deliver the particulate delivery system, optionally together with other additives that may alter the local microenvironment to promote stability and uptake, directly without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, e.g., almond oil, arachis oil, coconut oil, cottonseed oil, groundnut oil, corn germ oil, olive oil, castor oil, sesame seed oil, MIGLYOL™, glycerol, fractionated vegetable oils, mineral oils such as liquid paraffin, tetrahydrofurfuryl alcohol, polyethylene glycols, fatty acid esters of sorbitan, or mixtures of these substances. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances. Liquid formulations of a pharmaceutical composition of the disclosure that are suitable for oral administration can be prepared, packaged, and sold either in liquid form or in the form of a dry product intended for reconstitution with water or another suitable vehicle prior to use.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

For topical administration liquids, suspension, lotions, creams, gels, ointments, drops, suppositories, sprays and powders may be used. Conventional pharmaceutical carriers, aqueous, powder or oily bases, and thickeners can be used as necessary or desirable.

In other embodiments, the pharmaceutical composition can be prepared as a nutraceutical, i.e., in the form of, or added to, a food (e.g., a processed item intended for direct consumption) or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. The particulate delivery systems described herein are not exposed to high cooking temperatures for extended periods of time, in order to minimize degradation of the compounds.

Compositions for rectal or vaginal administration can be prepared by mixing a particulate delivery system with suitable non-irritating excipients or carriers such as cocoa butter, polyethylene glycol or a suppository wax, which are solid at ordinary room temperature, but liquid at body temperature, and therefore, melt in the rectum or vaginal cavity and release the particulate delivery system. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the active ingredient with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

A pharmaceutical composition of the disclosure can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the particulate delivery system suspended in a low-boiling propellant in a sealed container. Dry powder compositions may include a solid fine powder diluent such as sugar and are conveniently provided in a unit dose form. Low boiling propellants generally include liquid propellants having a boiling point below 65 degrees F. at atmospheric pressure. Generally, the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent, e.g., having a particle size of the same order as particles comprising the particulate delivery system.

Pharmaceutical compositions of the disclosure formulated for pulmonary delivery can also provide the active ingredient in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface-active agent, or a preservative such as methylhydroxybenzoate.

The formulations described herein as being useful for pulmonary delivery are also useful for intranasal delivery of a pharmaceutical composition of the disclosure. Another formulation suitable for intranasal administration is a coarse powder comprising the particulate delivery system. Such a formulation is administered in the manner in which snuff is taken, i.e., by rapid inhalation through the nasal passage from a container of the powder held close to the nares.

A pharmaceutical composition of the disclosure can be prepared, packaged, or sold in a formulation suitable for buccal administration. Such formulations can, for example, be in the form of tablets or lozenges made using conventional methods, and can, for example, comprise 0.1 to 20% (w/w) particulate delivery system, the balance comprising an orally dissolvable or degradable composition and, optionally, one or more of the additional ingredients described herein. Alternately, formulations suitable for buccal administration can comprise a powder or an aerosolized or atomized solution or suspension comprising the particulate delivery system.

Reference will now be made to specific examples illustrating the disclosure. It is to be understood that the examples are provided to illustrate exemplary embodiments and that no limitation to the scope of the disclosure is intended thereby.

### EXAMPLES

### Example 1: Production of Yeast Particles

YPs are typically 2-4 μm hollow and porous microparticles derived from Baker's yeast that are composed primarily of ˜80% 1→6-β branched, 1→3-β-glucan, 2-4% chitin and 40% mannan w/w.

Methods of preparing extracted yeast cell wall particles are known in the art, and are described, for example, in U.S. Pat. Nos. 4,992,540, 5,082,936, 5,028,703, 5,032,401, 5,322,841, 5,401,727, 5,504,079, 5,968,811, 6,444,448 B1, 6,476,003 B1, published U.S. applications 2003/0216346 A1, 2004/0014715 A1, and published PCT application WO 02/12348 A2, the disclosures of which are incorporated herein by reference.

A form of extracted yeast cell wall particles, referred to as “whole glucan particles” or “WPGs” (See U.S. Pat. Nos. 5,032,401 and 5,607,677) may be modified to facilitate improved retention and/or delivery of payload molecules. Such improvements feature trapping molecules and nanoparticles as well as pluralities of said trapping molecules and nanoparticles, formulated in specific forms to achieve the desired improved delivery properties. As used herein, a WGP is typically a whole glucan particle of >90% beta glucan purity.

### Preparation of Glucan Particles (GPs)

Glucan particles (GPs), also referred to herein as yeast glucan particles (“YGPs”), are a purified hollow yeast cell ‘ghost’ containing a rich β-glucan sphere, generally 2-4 microns in diameter. In general, glucan particles can be prepared from yeast cells by the extraction and purification of the alkali-insoluble glucan fraction from the yeast cell walls. The yeast cells can be treated with an aqueous hydroxide solution without disrupting the yeast cell walls, which digests the protein and intracellular portion of the cell, leaving the glucan wall component devoid of significant protein contamination, and having substantially the unaltered cell wall structure of β(1-6) and β(1-3) linked glucans. The 1,3-β-glucan outer shell provides for receptor-mediated uptake by phagocytic cells, e.g., macrophages, expressing 0-glucan receptors.

In certain exemplary embodiments, glucan particles are made as follows. Yeast particles (S. cerevisiae), Biorigin MOS55, are suspended in 1 liter of 1M NaOH and heated to 85° C. The cell suspension is stirred vigorously for 1 hour at this temperature. The insoluble material containing the cell walls is recovered by centrifuging. This material is then suspended in 1M NaOH, heated, and stirred vigorously for 1 hour. The suspension is allowed to cool to room temperature and the extraction is continued for a further 16 hours. The insoluble residue is recovered by centrifugation. This material is finally extracted in water brought to pH 4.5 with HCl. The insoluble residue is recovered by centrifugation and washed three times with water, isopropanol, and acetone. The resulting slurry is placed in glass trays and dried under reduced pressure to produce a fine white powder.

### Preparation of Glucan Lipid Particles (GLPs)

GLPs retain some of the yeast cellular lipid content, which creates a more hydrophobic inner cavity ideal for loading of hydrophobic payloads. GLPs are prepared by modifying the method of preparation of GPs described above. For preparation of GLPs, washing with isopropanol and acetone is eliminated and instead the insoluble residue recovered by centrifugation is washed three times with water. The particles are dried by lyophilization or spray drying.

Commercial Yeast Particles (YPs)

Yeast particles (YPs) were purchased from Biorigin (Louisville, KY, USA) or LeSaffre (Marcq-en-Barceul, France). These YPs contained sufficient amounts of lipids to provide for a hydrophobic reservoir that attracts hydrophobic payloads to diffuse into the center of the particle accomplishing loading.

Whole Glucan Particles (WGPs)

A more detailed description of processes for preparing WPGs can be found in U.S. Pat. Nos. 4,810,646, 4,992,540, 5,028,703, 5,607,677, and 5,741,495 (incorporated herein by reference). For example, U.S. Pat. No. 5,028,703 discloses that yeast WGP particles can be produced from yeast strain R4 cells in fermentation culture. The cells are harvested by batch centrifugation at 8000 rpm for 20 minutes in a Sorval RC2-B centrifuge. The cells are washed twice in distilled water in order to prepare them for the extraction of the whole glucan. The first step involved resuspending the cell mass in 1 liter 4% w/v NaOH and heating to 100° C. The cell suspension is stirred vigorously for 1 hour at this temperature. The insoluble material containing the cell walls is recovered by centrifuging at 2000 rpm for 15 minutes. This material is suspended in 2 liters, 3% w/v NaOH and heated to 75° C. The suspension is stirred vigorously for 3 hours at this temperature. The suspension is then allowed to cool to room temperature and the extraction can be continued for a further 16 hours. The insoluble residue is recovered by centrifugation at 2000 rpm for 15 minutes. This material is finally extracted in 2 liters, 3% w/v NaOH brought to pH 4.5 with HCl, at 75° C. for 1 hour. The insoluble residue is recovered by centrifugation and washed three times with 200 milliliters water, once with 200 milliliters dehydrated ethanol, and twice with 200 milliliters dehydrated ethyl ether. The resulting slurry is placed on petri plates and dried.

Varying degrees of purity of glucan particles are achieved by modifying the extraction/purification process. In general, these GPs are on the order of 80-85% pure on a w/w basis of beta glucan and, following the introduction of payload, trapping, or other components, become of a slightly lesser “purity.” In exemplary embodiments, GPs are <90% beta glucan purity.

### Preparation of YCP Particles

Yeast cells (Rhodotorula sp.) derived from cultures obtained from the American Type Culture Collection (ATCC, Manassas, VA) are aerobically grown to stationary phase in YPD at 30° C. Rhodotorula sp. cultures available from ATCC include Nos. 886, 917, 9336, 18101, 20254, 20837 and 28983. Cells are harvested by batch centrifugation at 2000 rpm for 10 minutes. The cells are then washed once in distilled water and then resuspended in water brought to pH 4.5 with HCl, at 75° C. for 1 hour. The insoluble material containing the cell walls is recovered by centrifuging. This material is then suspended in 1 liter, 1M NaOH and heated to 90° C. for 1 hour. The suspension is allowed to cool to room temperature and the extraction is continued for a further 16 hours. The insoluble residue is recovered by centrifugation and washed twice with water, isopropanol, and acetone. The resulting slurry is placed in glass trays and dried at room temperature to produce 2.7 g of a fine light brown powder.

In alternative embodiments, YGPs, e.g., activated YGPs, are grafted with chitosan on the surface, for example, to increase total surface chitosan. Chitosan can further be acetylated to form chitin (YGCP), in certain embodiments. Such particles have equivalent properties in vivo when detected by the immune system of a subject or patient.

### Preparation of YGMP Particles

S. cerevisiae (100 g Fleishman's Baker's yeast) was suspended in 1 liter 1M NaOH and heated to 55° C. The cell suspension was mixed for 1 hour at this temperature. The insoluble material containing the cell walls was recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with 1000 milliliters water, four times with 200 milliliters dehydrated isopropanol and twice with 200 milliliters acetone. The resulting slurry was placed in a glass tray and dried at room temperature to produce 12.4 g of a fine, slightly off-white powder.

S. cerevisiae (75 g SAF-Mannan) was suspended in 1 liter water and adjusted to pH 12-12.5 with 1M NaOH and heated to 55° C. The cell suspension was mixed for 1 hour at this temperature. The insoluble material containing the cell walls was recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with water, dehydrated isopropanol, and acetone. The resulting slurry was placed in a glass tray and dried at room temperature to produce 15.6 g of a fine slightly off-white powder.

### Preparation of Glucan Mannan Lipid Particles (GMLPs)

GMLPs were prepared by the procedure described above for preparation of YGLPs but without the steps requiring washing with isopropanol and acetone.

### Example 2: Ensilication of Payload in Glucan Particles

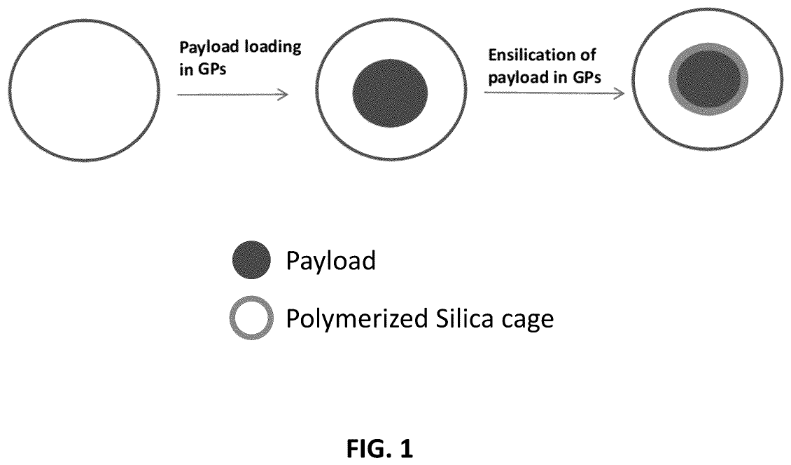

FIG. 1 shows a schematic diagram for ensilicating payloads inside YPs according to an exemplary embodiment of the disclosure.

The chemical reactions resulting in polymerization and hydrolysis of tetraethylorthosilicate (TEOS) are described by Buckley et al. (Buckley, A. M., & Greenblatt, M. (1994). The sol-gel preparation of silica gels. Journal of Chemical Education, 71(7), 599-602).

A procedure for the ensilication of payloads with GPs was developed by modifying the procedure described by Chen et al. (Chen, Y-C; et al. Thermal stability, storage and release of proteins with tailored fit in silica. Scientific Reports, 2017, 7, 46568). Partially prepolymerized TEOS of a particular size capable of diffusing into hydrated YPs was used. The TEOS could dissolve the payload and polymerize in situ within the hollow YP cavity to trap the payload molecules in a silicate glass to provide increased stability. The speed of the ensilication reaction was decreased and the reaction was carried out in half hydrodynamic volume. These modifications ensured that the ensilicated payload remained inside the YPs. In this procedure no polymerization occurred outside the particles. As polymerization outside the YPs leads to particle clumping, the instant modified procedure ensured that YPs remained as individual particles without aggregating. The TEOS polymerization on the interior of YPs ensured that payload trapping/ensilication occurred inside the YPs instead of on the outside of YPs.

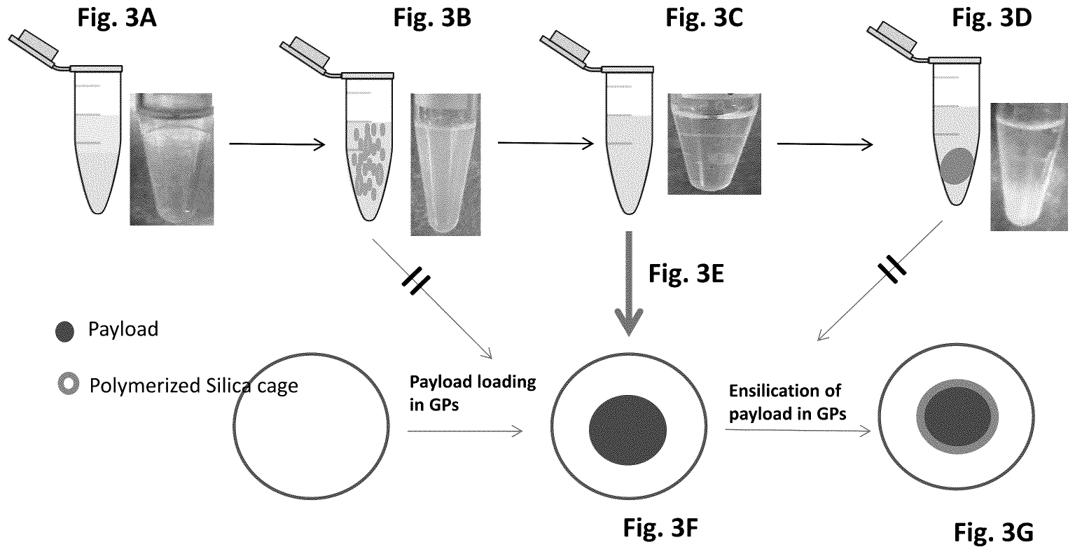

Ensilication of payloads within GPs involves several steps which are shown in FIGS. 3A-G. Initial TEOS (top phase, d=0.93 g/mL) and aqueous (acid or base) solution (bottom phase) (FIG. 3A) when mixed formed a TEOS-aqueous emulsion composed of pre-polymerized TEOS far from gel point (FIG. 3B). Upon further incubation, the TEOS-aqueous solution formed pre-polymerized TEOS close to gel point (FIG. 3C), which upon further incubation formed silica gel as the TEOS fully polymerized (FIG. 3D). To ensilicate payloads in GPs, a TEOS-aqueous solution close to gel point was loaded into GPs containing payloads. The TEOS gel then fully polymerized inside GPs, thereby forming a silica cage around the payload molecules within the GPs (FIG. 3G).

### Example 3: Ensilication of Bovine Serum Albumin (BSA, MW=66.5 kD) within GPs

The efficiency of ensilication of BSA within GPs was tested. 20 μL of a 25 mg rhodamine labeled BSA (rBSA)/mL aqueous solution was added to a dry sample of 5 mg fluorescein labeled GPs (fGPs). The samples were mixed, incubated at room temperature for 30 minutes, frozen and lyophilized to remove solvent. The loading cycle was repeated by adding 10 μL of water to the dry fGP rBSA sample to draw payload within the matrix of the GP shell into the hollow GP cavity, the sample was mixed and lyophilized to remove solvent.

TEOS was mixed with 0.01 M HCl solution (pH 2). The mixture was mixed for 1 hour to achieve TEOS prepolymerization and formation of a single aqueous phase in which the TEOS was close to its gel point. Solutions with different ratios of TEOS and 0.01 M HCl were prepared to target TEOS:rBSA weight ratios from 0 to 24.9. The pre-polymerized TEOS mixture was added to the dry fGP-rBSA pellet (20 μL/5 mg fGP). The sample was mixed and incubated at room temperature for 1 hour.

The ensilicated fGP-rBSA samples were washed three times with 0.9% saline. The ensilicated fGP rBSA pellets were evaluated by microscopy to assess efficient trapping of rBSA within the cavity of fGPs, and percent protein encapsulation was quantified by SDS-PAGE. Encapsulation of rBSA within fGPs was visualized by brightfield and fluorescent microscopy overlays of the fGP rBSA [SiO₂]ₙ formulation.

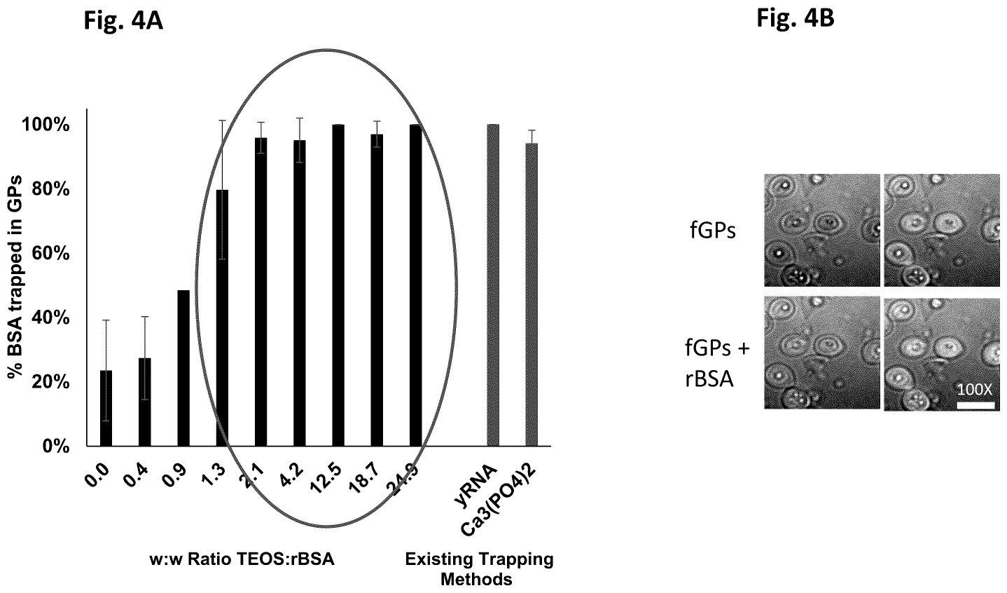

TEOS pre-polymerized at pH 2 with TEOS:rBSA ratios of 1.3 to 24.9 ensilicated and trapped rBSA within GPs at high efficiency (>80%) comparable to conventional methods of trapping payload within GPs using yRNA or Ca₃(PO₄)₂ (FIG. 4A). Fluorescent micrography confirmed that the rBSA was encapsulated within GPs (FIG. 4B).

Ensilication of rBSA in GPs Using a Fluorescent Silica Shell (TEOS/f-APTES)

Fluorescent precursor for ensilication (f-APTES) was synthesized by reaction of 3-amino-propyl-triethoxysilane with fluorescein-isothiocyanate (FITC) in ethanol at room temperature under nitrogen atmosphere. A mixture of 0.01% f-APTES and 99.99% TEOS was used for ensilication.

Fluorescent rBSA was loaded in GPs as described above. The GPs used were non-fluorescent. The fAPTES/TEOS ensilication mixture was prepared in 0.01 M HCl as described above. Encapsulation of rBSA within GPs was visualized by brightfield and fluorescent microscopy overlays.

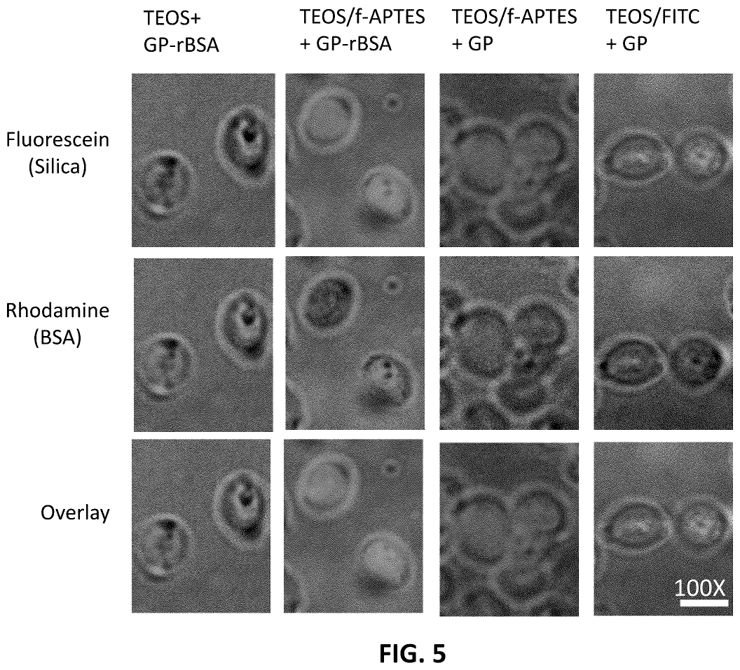

FIG. 5 shows brightfield and fluorescent microscopy overlays of GPs with or without rBSA and fluorescent silica shell (TEOS/f-APTES) and confirms that fully polymerized silica shells were located inside GPs.

### Example 4: Interaction of Ensilicated fGP-BSA Samples with Macrophages

Macrophages Efficiently Uptake fGP BSA [SiO₂]ₙ Via Phagocytosis

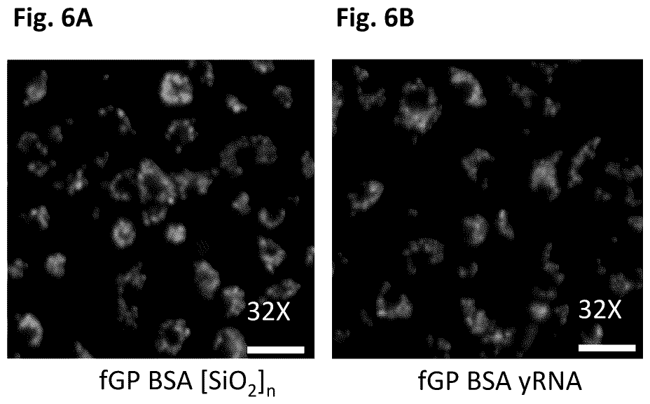

Sterile fGP samples in 0.9% saline were diluted at a concentration of 1×10⁸ particles/mL. 10 μL of fGP samples (1×10⁶ particles) were added to wells in a 96-well plate containing 1×10⁵ B6 macrophage cells per well (fGP:cell ratio of 10:1) in complete Dulbecco's Modified Eagle Medium (DMEM). The plates were incubated at 37° C., 5% CO₂ for 24 hours and then particle uptake was assessed by tracking of fGPs by fluorescent microscopy. FIG. 6 shows that fGPs containing ensilicated BSA were phagocytosed with equal efficiency as the control fGPs containing BSA trapped via conventional methods using yRNA.

Ensilicated GP-BSA Samples are not Toxic to Macrophages

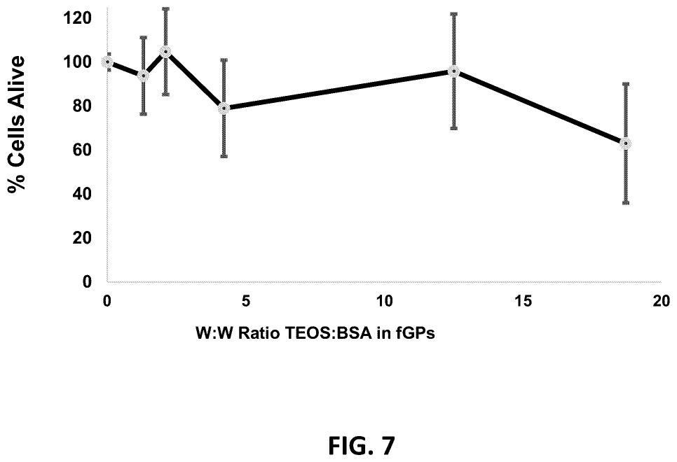

Sterile ensilicated fGP BSA samples in 0.9% saline were added to wells in a 96-well plate containing B6 macrophage cells. The fGPs were added at a ratio of 10:1 particles:cell. The plates were incubated at 37° C., 5% CO₂ for 24 hours. ALAMAR BLUE™ solution was added (10 μL per well) and the plate was incubated for 30 minutes at 37° C., 5% CO₂. ALAMAR BLUE™ fluorescence was measured (excitation wavelength=530 nm, emission wavelength=590 nm). Fluorescence response is dependent on the reduction of the ALAMAR BLUE™ indicator by metabolically active cells and thus, is a measure of cell number and viability. The percent of live cells was calculated from the fluorescence response of the sample relative to the response of control wells containing buffer (PBS) or empty GPs. FIG. 7 shows that at low W:W ratio of TEOS:BSA, fGPs exhibited very little toxicity on the macrophage cell line. Even at higher TEOS:BSA W:W ratios, more than 60% of the cells were viable after exposure to fGPs containing ensilicated BSA. The results show that GPs containing ensilicated payloads were not very cytotoxic to macrophages.

Ensilicated BSA is Efficiently Released Inside B6 Macrophage Cells

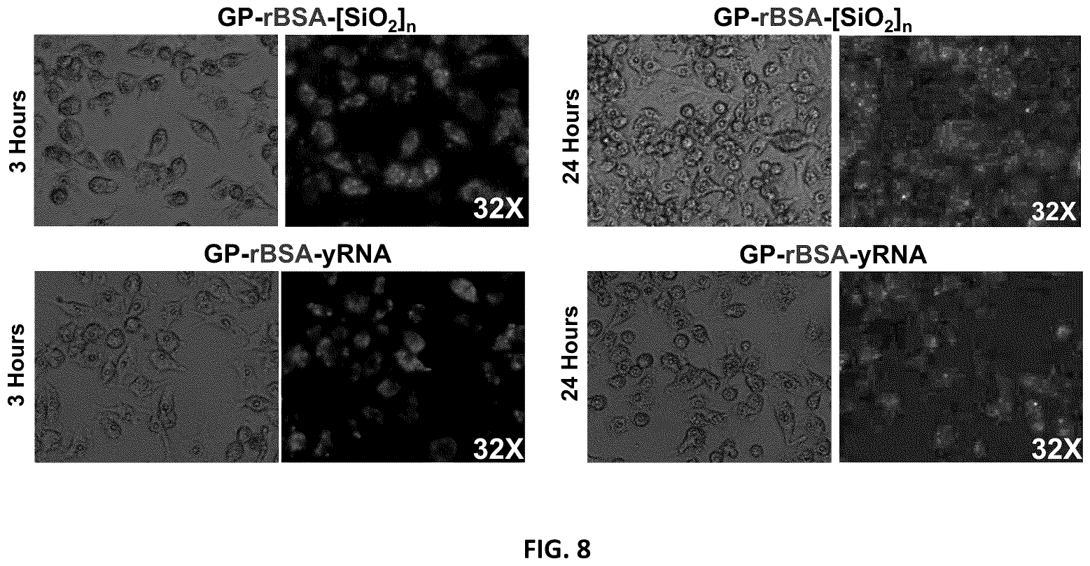

Following uptake of GPs containing ensilicated rBSA, release of rBSA was tracked as described above using rhodamine labeled BSA to track the location of the protein at the 3 hour and 24 hour time points following GP uptake by B6 cells. GPs encapsulating BSA trapped with yRNA were used as control. FIG. 8 shows that both ensilicated BSA and BSA trapped with yRNA were located within GPs at the 3 hour time point, but were released within macrophage cells by the 24 hour time point. This result confirmed that ensilicated payload could be efficiently released inside macrophage cells.

### Example 5: Optimization of Ensilication of Bovine Serum Albumin (BSA, MW=66.5 kD)

Effect of pH on Ensilication Efficiency

TEOS was pre-polymerized as described above. Solutions of different concentrations of HCl and NaOH were used to generate different pH conditions. TEOS was allowed to pre-polymerize for 1, 6 or 20 hours prior to loading it into GPs containing BSA payload.

Acid catalyzed TEOS forms long, linear polymers that easily entangle and gel. Base catalyzed TEOS forms short, branch clusters that may not trap payload with high efficiency. (Buckley et al., J. Chem. Ed. 1994). The percent of BSA trapped in GPs was measured as described above.

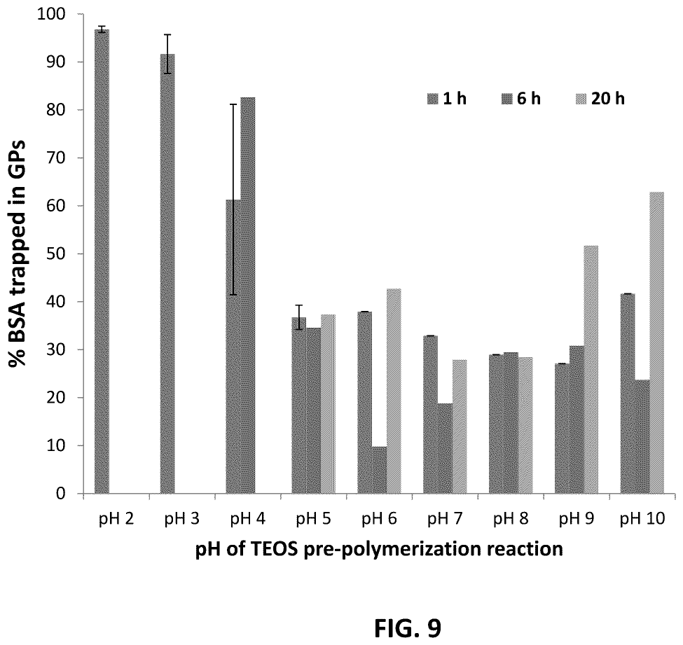

FIG. 9 shows the BSA trapping efficiency of TEOS pre-polymerized at various pH values for varying amounts of time prior to loading it into GPs containing BSA payload. Results show that 1 hour pre-polymerization time at pH 2 or pH 3 was the most optimal condition for achieving highest BSA trapping efficiency. At pH of 4, a high efficiency was achieved by increasing the pre-polymerization time to 6 hours. At pH of 9 or 10, a moderate trapping efficiency (50-70%) was achieved by increasing pre-polymerization time to 20 hours. At pH values of 5-8, a very slow hydrolysis/condensation of TEOS occurred which reduced BSA trapping efficiency to below 40% at all pre-polymerization time points used.

Ensilication of Buffered GP-BSA with TEOS Pre-Polymerized at pH2

The effect of the pH of GP-BSA and TEOS mixture on ensilication efficiency was tested. TEOS mixture was pre-polymerized at a pH 2 and mixed with GP-BSA to achieve a TEOS:BSA w/w ratio of 12.5:1. The various experimental conditions tested are summarized in Table 1.

TABLE 1
Experimental conditions used for testing ensilication
efficiency of TEOS pre-polymerized at pH 2 with
GP-BSA prepared in water or buffer.

| | | Post-loading | | |
|---|---|---|---|---|
| | | Resuspension | TEOS | |
| Experimental | GP-BSA | of GP-BSA in | Mixture at | Mixing |
| conditions | loading in | a buffer at pH | pH | Procedure |
| A | water | — | pH 2 | Resuspend GP- |
| (pH 2) | | | | BSA in TEOS |
| | | | | mixture |
| B | Water | pH 7 | pH 2 | Add TEOS |
| (pH 2/pH 7 | | | | mixture to GP- |
| Sequential | | | | BSA |
| load) | | | | |
| C | PBS* pH 7 | — | pH 2 | Resuspend GP- |
| (pH 2/ pH 7 | | | | BSA in TEOS |
| co-load) | | | | mixture |
| D | PBS pH 7 | — | PBS pH 7 | Resuspend GP- |
| (pH 7) | | | | BSA in TEOS |
| | | | | mixture |

*PBS = Phosphate buffered saline

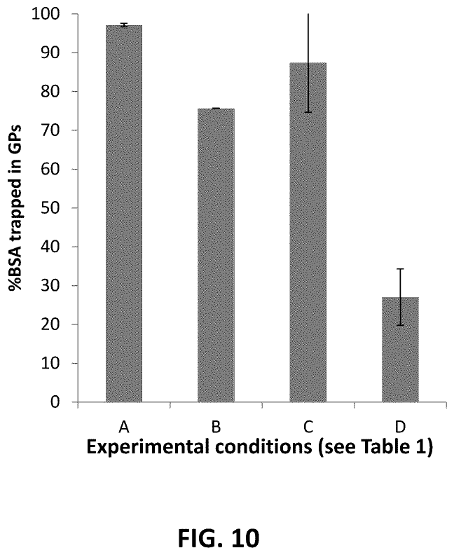

FIG. 10 shows that ensilication efficiency was the highest when GP-BSA was prepared in water and resuspended in TEOS mixture prepolymerized at pH 2.

Effect of Loading of BSA in GPs in the Presence of 6M Urea on Ensilication Efficiency

To test whether proteins could be loaded in GPs in the presence of denaturing conditions, BSA was loaded into GPs in the presence of water or in high concentration of urea (6M). The ensilication reaction was carried out as described above using TEOS prepolymerized at pH 2. The w/w ratio of TEOS:BSA used was 12.5:1.

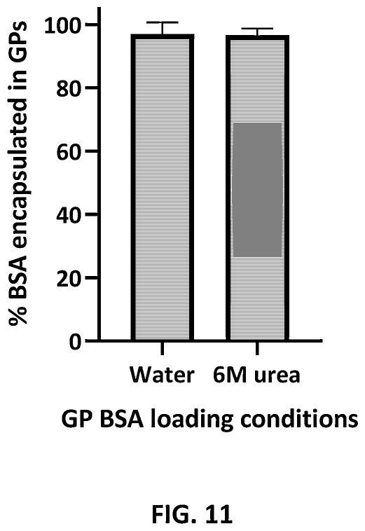

FIG. 11 shows that when BSA was loaded in GPs in the presence of 6M urea, the ensilication efficiency improved.

Effect of Loading of BSA in GPs Under High Salt Concentration on Ensilication Efficiency

To test whether proteins could be loaded in GPs under high salt concentrations, BSA was loaded into GPs in the presence of water or sodium chloride (NaCl) at a concentration of 1M or 5M. Ensilication reaction was carried out as described before using TEOS prepolymerized at pH 2. The w/w ratio of TEOS:BSA used was 12.5:1.

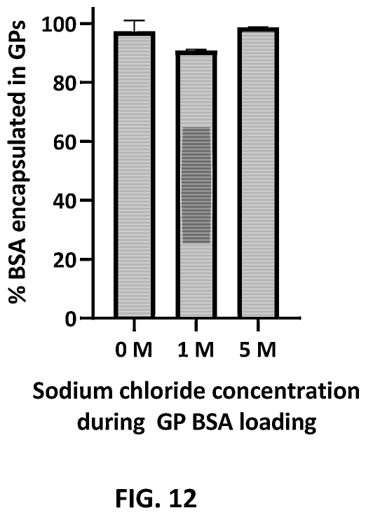

FIG. 12 shows that ensilication efficacy was not impacted when BSA was loaded in GPs in the presence of high concentrations of sodium chloride.

Effect of Strong or Weak Acids and Incubation Conditions on Ensilication Efficiency

BSA was loaded in GPs as described above. TEOS was prepolymerized as described before but at pH 4 using a strong (HCl) or a weak (acetic) acid. After mixing the prepolymerized TEOS with GP-BSA, the mixture was allowed to incubate for 1, 4 or 18 hours at a temperature of 4° C. or 23° C. Percent of BSA trapped in GPs was calculated as described above.

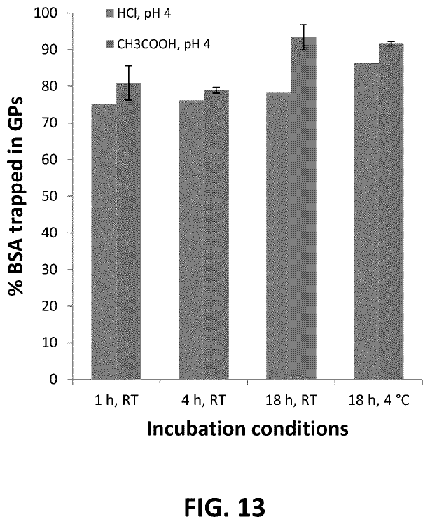

FIG. 13 shows that ensilication efficiency was high when TEOS was prepolymerized in a weak acid and the TEOS-GP-BSA mixture was incubated at low temperatures for prolonged periods. These results indicate that proteins unstable at room temperature or in strong (pH 2) acids can still be efficiently encapsulated and ensilicated within GPs using alternative methods.

### Example 5: Encapsulation of Two Different Payloads in GPs

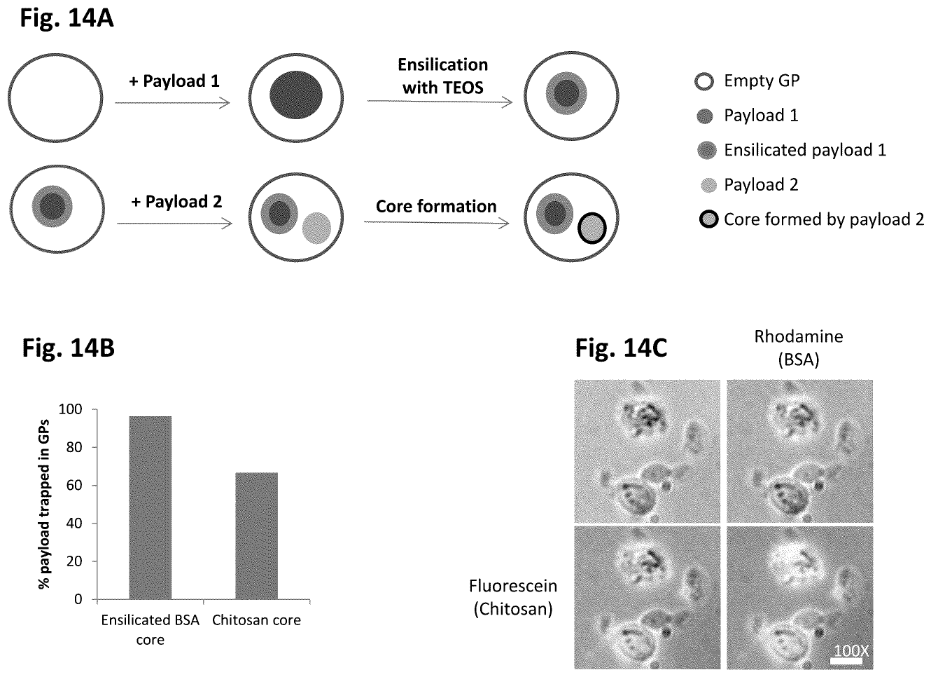

To test whether a second payload could be added to GPs after ensilication of the first payload, chitosan was added to GPs after GPs were loaded with BSA. FIG. 14A shows the schematic of the procedure to load two payloads in GPs. GPs were loaded with rhodamine labeled BSA (rBSA) as described above. GPs loaded with rBSA were then subjected to the ensilication protocol as described above. After ensilication of rBSA, fluorescent labeled chitosan (f-chitosan) was loaded in half hydrodynamic volume (5 μL/mg GP) using a solution of f-chitosan at pH 5, lyophilized, and then phosphate buffer (pH 7) was added to precipitate the f-chitosan core.

FIG. 14B shows that both BSA and chitosan could be efficiently trapped within GPs following the sequential loading procedure shown in FIG. 14A. FIG. 14C shows that both BSA and chitosan payloads were trapped within GPs. This data confirms after encapsulation of a first payload via ensilication, a second payload can be encapsulated within GPs.

### Example 6: Ensilication with Tetraethylorthogermanate (TEOG)

The ensilication efficiency of tetraethylorthogermanate (TEOG) was compared with TEOS. GPs were loaded with BSA as described above. TEOG was prepolymerized by the same process as described above for TEOS at pH 2 and pH 10. GP-BSA was incubated with prepolymerized TEOG to promote ensilication of BSA within GPs. TEOS was used as control for comparison of trapping efficiency.

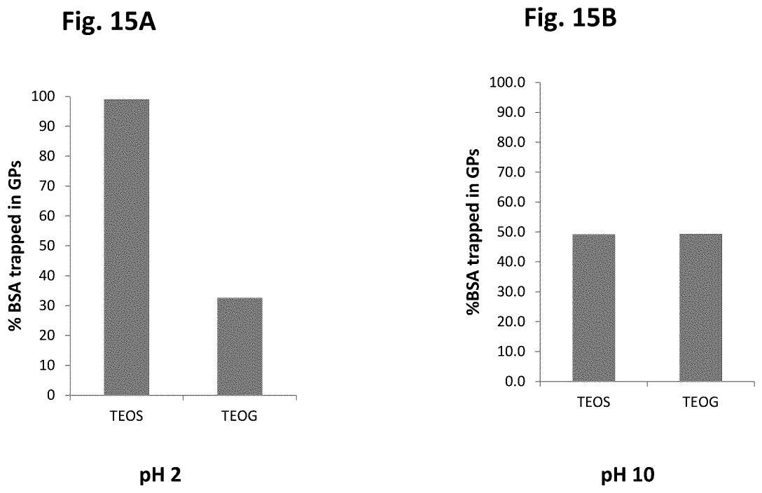

FIGS. 15A and B show the BSA trapping efficiencies of TEOG and TEOS at pH 2 and pH 10, respectively. At pH 2, TEOG gelled rapidly, in less than 1 minute, thus preventing efficient loading of TEOG in GPs. However, at pH 10 TEOG, gels more slowly formed over 10-20 minutes. Thus, the best trapping efficiency was achieved with TEOG at pH 10.

### Example 7: Ensilicated Cores for Layer-by-Layer (LbL) Payload Absorption

Layer-by-Layer Adsorption of Charged Polymers on GP Ensilicated Cores

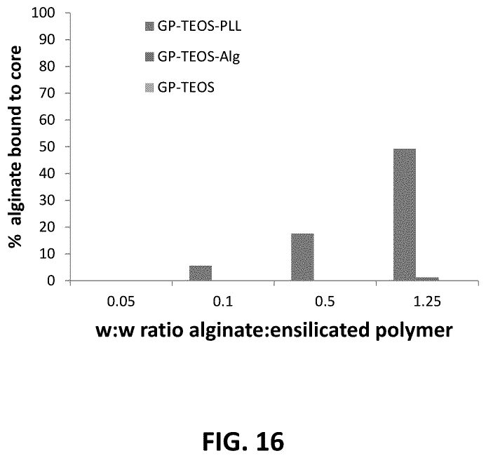

The ability of anionic alginate to adsorb on GP partially ensilicated PLL or alginate cores was tested. Anionic fluorescent alginate was used. Data presented in FIG. 16 shows that anionic fluorescent alginate bound only to a GP cationic ensilicated PLL cores. The percent of alginate bound to the core increased as the w/w ratio of alginate:ensilicated polymer increased.

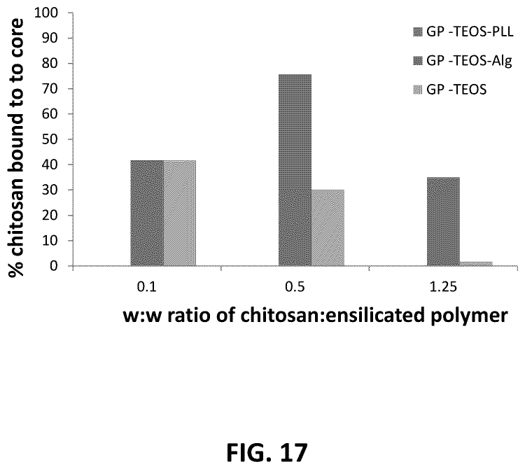

The ability of cationic fluorescent chitosan (MW 15 kD) to adsorb on partially ensilicated GP PLL or alginate ensilicated cores were tested. Data presented in FIG. 17 shows that cationic fluorescent chitosan bound more efficiently to just GP TEOS or GP anionic alginate cores partially ensilicated by TEOS.

### Example 8: Stability of BSA Ensilicated within GPs

The ability of the ensilication process to protect BSA from thermal denaturation and enzymatic digestion was tested.

Stability of BSA in the Presence of Proteases

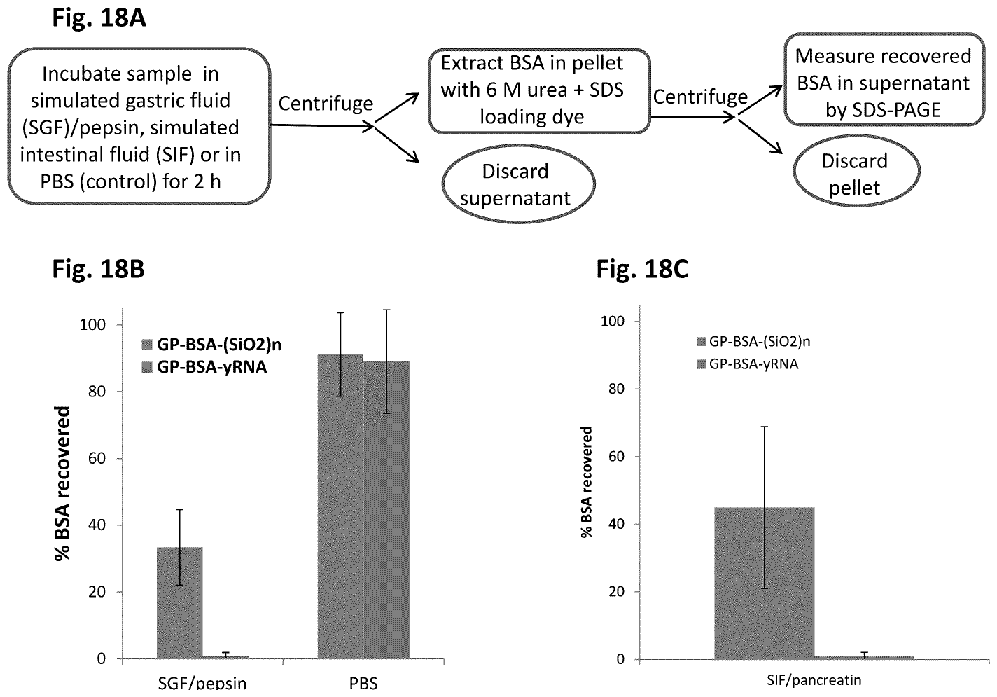

Samples of GPs containing ensilicated BSA were incubated with simulated gastric fluid (SGF), pepsin, simulated intestinal fluid (SIF) or pancreatin for 2 hours. Samples were centrifuged and supernatants were discarded. The BSA in the pellets were extracted with 6M urea and SDS PAGE loading dye. The extracted BSA was centrifuged. The precipitated protein in the pellets was discarded and BSA remaining in the supernatants was quantified by SDS-PAGE. FIG. 18A shows a flowchart summarizing the steps for extraction and quantification of BSA after incubation of GP containing ensilicated BSA with SGF/pepsin.

Ensilicated BSA was more resistant to enzymatic digestion compared to BSA encapsulated via yRNA trapping when exposed to SGF/pepsin (FIG. 18B) or SIF/pancreatin (FIG. 18C).

Improved Stability of Ensilicated GP-BSA with Eudragit® Coating

EUDRAGIT® L100 is a commonly used polymethacrylate-based coating polymer available from Evonik Industries. EUDRAGIT® L100 coats are water insoluble at pH<5.5, but dissolve at pH above 5.5. A EUDRAGIT® coating layer is useful in preventing drug release and degradation in the stomach.

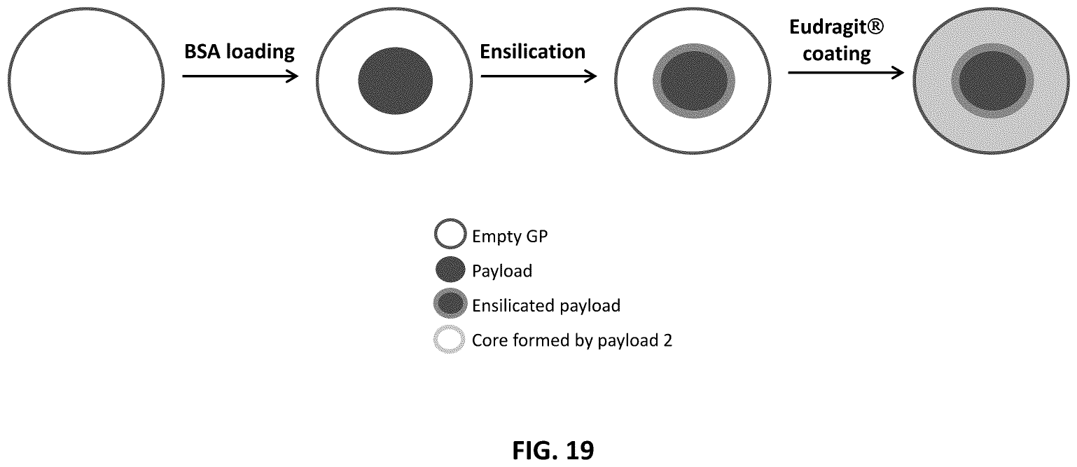

The ability of a EUDRAGIT® coat to further enhance the stability of BSA trapped in GPs was tested. GPs containing either ensilicated BSA or yRNA trapped BSA were coated with EUDRAGIT®. FIG. 19 shows a schematic of the procedure for preparation of EUDRAGIT® coated ensilicated payload within GPs according to exemplary embodiments.

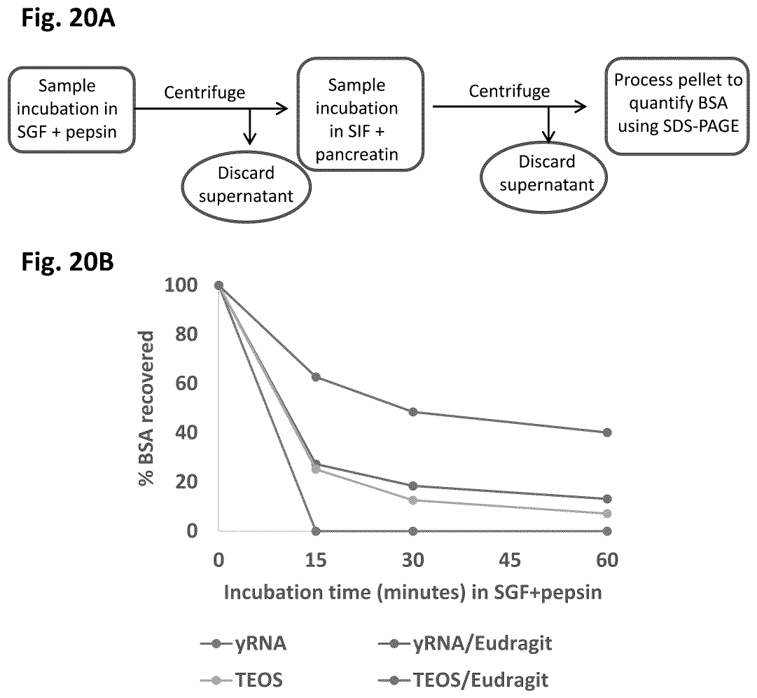

Samples of GPs containing BSA coated with EUDRAGIT® were incubated with SGF/pepsin for 2 hours. Samples were centrifuged and supernatants discarded. Samples were further incubated with SIF/pancreatin for 2 hours. Samples were centrifuged and supernatants discarded. The BSA in the pellets was extracted with 6M urea and SDS loading dye. The extracted BSA was centrifuged. The precipitated protein in the pellets was discarded and BSA remaining in the supernatant was quantified by SDS-PAGE. FIG. 20A shows a flowchart summarizing the steps for extraction and quantification of BSA after incubation of GP containing EUDRAGIT® coated ensilicated BSA with SGF/pepsin followed by SIF/pancreatin. FIG. 20B shows percent of BSA remaining in GPs after exposure of various GP samples to enzymatic digestion.

The results confirm that the combination of ensilication and EUDRAGIT® coating of BSA in GPs provided the maximum protection from enzymatic digestion.

Thermal Stability of BSA Ensilicated in GPs

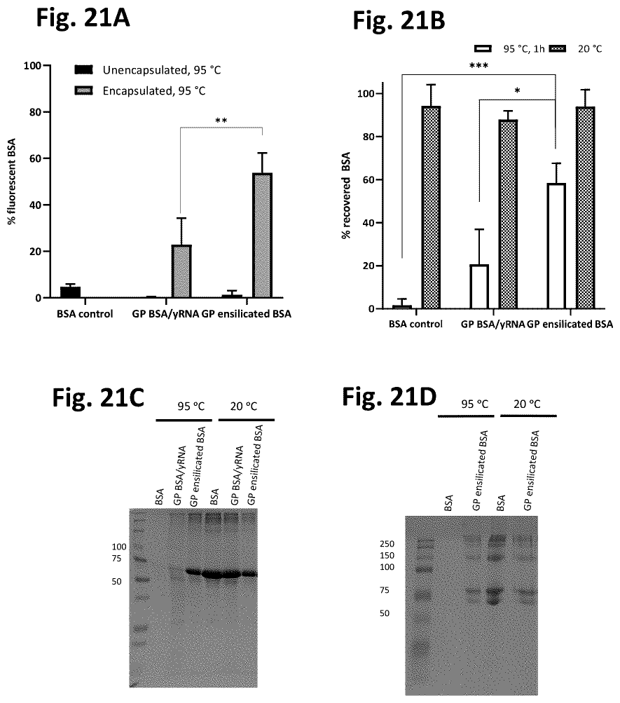

BSA was loaded in GPs as described above either with yRNA or with ensilication using TEOS. GPs containing ensilicated BSA were incubated in PBS at 95° C. for 1 hour or at 20° C. (control). Samples were centrifuged to separate unencapsulated BSA released in the supernatants and encapsulated BSA remaining in the pellets. The BSA was quantified by fluorescence and SDS-PAGE densitometry. FIG. 21 shows the encapsulated and unencapsulated BSA after heat treatment quantified by fluorescence (FIG. 21A) or SDS-PAGE densitometry (FIG. 21B). FIG. 21C depicts the SDS-PAGE showing recovered protein, and (D) native gel showing intact BSA monomer, dimer and trimer recovery from GP ensilicated BSA.

### Example 9: Plug Seal Approach: Silicate Capped GPs

Production of Silicate Capped YPs

To prepare silicate capped GPs, the method disclosed by T. E. Wagner was adapted (Wagner, T. E.; Vaccine Delivery Systems Using Yeast Cell Wall Particles, US patent application publication No. US20170007688A1).

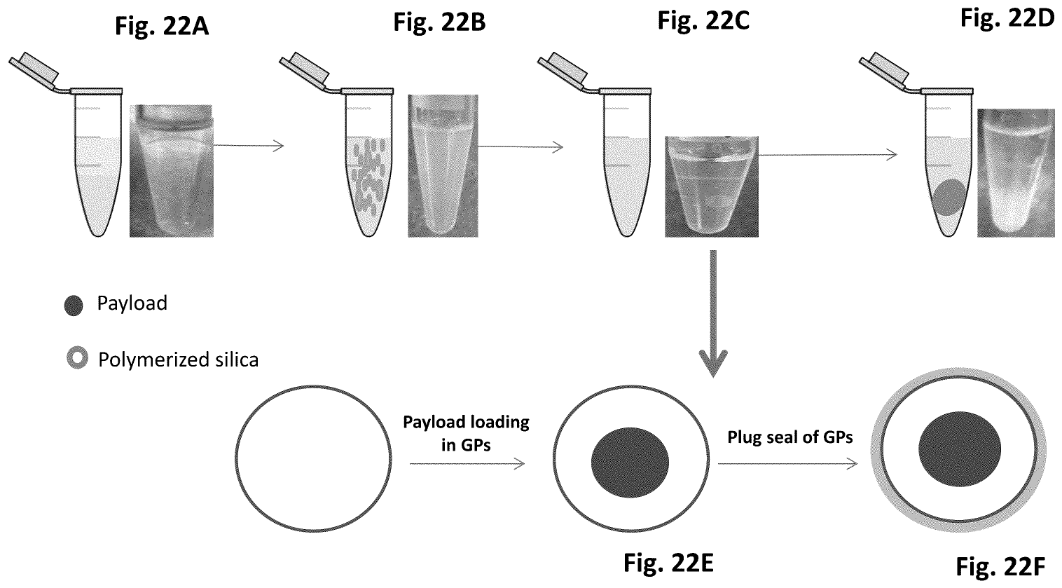

A procedure for production of silicate capped GPs according to exemplary embodiments involves several steps which are shown in FIG. 22. An initial TEOS (top phase, d=0.93 g/mL) and aqueous NH₄OH solution (bottom phase) (FIG. 22A), when mixed, formed a TEOS-NH₄OH aqueous emulsion composed of pre-polymerized TEOS far from gel point (FIG. 22B). Upon further incubation, the TEOS-aqueous solution formed pre-polymerized TEOS close to gel point (FIG. 22C), which rapidly (<10 minutes) fully polymerized to form a silica gel (FIG. 22D). When the TEOS-NH₄OH aqueous solution close to gel point (FIG. 22C) was incubated with GPs containing payloads (FIG. 22E), the very rapid polymerization of TEOS in basic NH₄OH prevented TEOS from diffusing inside GPs, and instead resulted in polymerization of TEOS on the GP surface, resulting in plug sealed or silicate capped GPs (FIG. 22F).

GPs loaded with fluorescent BSA (fBSA) were plug sealed (silicate capped) by the above protocol using 10% NH₄OH as catalyst. GPs containing ensilicated fBSA were also prepared as described before at the optimal TEOS polymerization conditions with 0.01 M HCl (pH 2) for use as control. Percent of BSA trapped in GPs was measured as described above. The plug seal protocol known in the art at the time of filing requires a large volume (1 mL per mg GP) and use of ethanol to prevent premature release of BSA from GPs. In contrast, a key difference of methods disclosed herein is that ensilication can be performed in half hydrodynamic volumes (5 μL per mg GP), trapping the BSA payload inside the GPs.

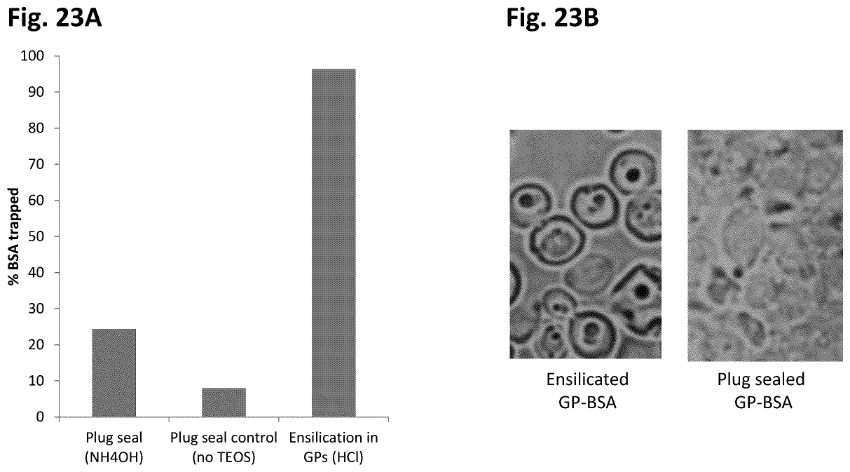

FIG. 23A shows the percent of BSA trapped in GPs in control (no TEOS), ensilication, and plug seal approach. Data indicates that ensilication of BSA within GPs was a significantly better trapping strategy as nearly 100% of the BSA was trapped in GPs using the ensilication approach. The plug seal approach was much less efficient in retaining BSA within GPs, as it resulted in trapping of just over 20% of BSA. FIG. 23B shows fluorescent micrographs indicating location of fBSA. The images also show that plug sealed GPs clumped together due to rapid silica gel formation.

In Vitro Leaking Assay

To detect leaking of payload from plug sealed or silicate capped YPs, an in vitro leaking assay was performed. Dry YPs were loaded with a payload (e.g., peptide or fluorescent albumin) and then were resuspended in ethanol (1 mg YP/mL). TEOS (100 μL) and 10% aqueous ammonia solution (100 μL) were added to the YP suspension. The mixture was incubated 15 min at room temperature. Silicate capped YPs were washed with ethanol and stored in ethanol at 4° C.

An in vitro leaking assay was performed on silicate capped YPs containing fluorescent albumin by the following procedure. Silicate capped YPs containing fluorescent albumin were incubated in PBS. Samples of supernatant were collected at 0, 1 and 2 hour time points. Fluorescent albumin in supernatant was measured. After 1 hour, uncapped YPs leaked 24.73% of fluorescent payload and silicate capped YPs leaked 15.81% of fluorescent payload. After 2 hours, uncapped YPs leaked 16.6% and silicate capped YPs leaked 6.65% of payload. The total loss of payload due to leakage was 41.33% in uncapped YPs but only 22.46% in capped YPs. While uncapped YPs retained 58.67% of the payload protein, capped YPs retained significantly more, 77.54% of the payload. In comparison, a YP ensilicated f-BSA control sample efficiently retains the ensilicated payload better than the capped YPs with <1% f-BSA detected in the supernatant after 2 h incubation or >99% of the payload retained in the YPs.

### Example 10: BSA Ensilication in GPs with NH₄OH as a Catalyst

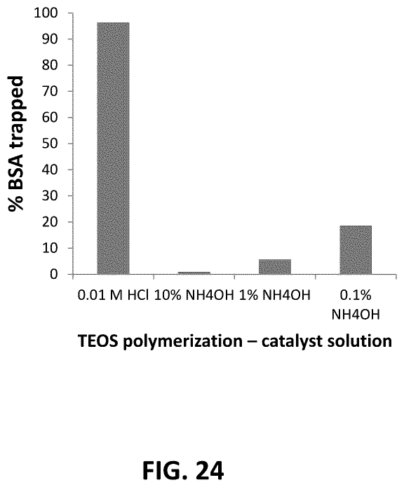

The ability of NH₄OH to catalyze ensilication of BSA within GPs was tested. NH₄OH was added as a polymerization catalyst at a concentration of 0.1%, 1%, and 10% to the mixture of GP-BSA and prepolymerized TEOS. The percent of BSA trapped in GPs was measured as described above and is shown in FIG. 24. Rapid gel formation of TEOS in ammonium hydroxide prevented efficient GP loading of prepolymerized TEOS.

### Example 11: Use of Ensilicated Payloads in GPs as Vaccines

Ensilicated OVA and MSA in GPs

GPs were loaded with chicken ovalbumin (OVA) payload. OVA was either trapped with yRNA or ensilicated with TEOS by the procedure described before.

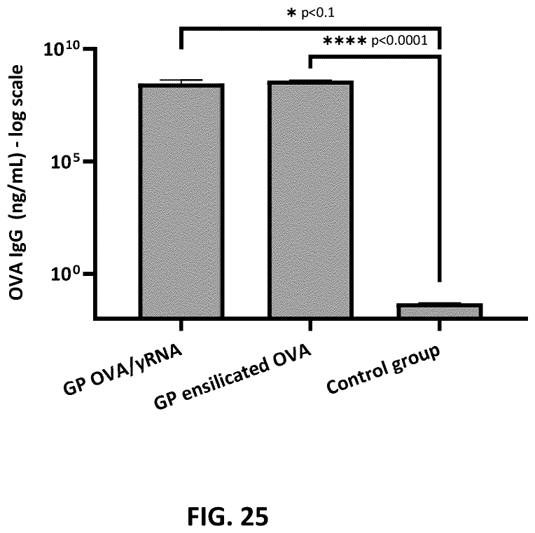

Mice were immunized by subcutaneous (SC) administration of GPs containing OVA payload trapped within GPs via ensilication or yRNA. Anti-OVA antibodies in mouse serum were measured by ELISA analysis of OVA IgG at 10⁷ fold dilution. Mice developed high IgG antibody titers regardless of the method used to trap antigen payload (FIG. 25). The results show that ensilicated GP-OVA-MSA served as effective a vaccine as the control GP-OVA-MSA/yRNA vaccine.

Ensilicated GP Cryptococcal Vaccine Antigens

Meningitis due to Cryptococcus neoformans is responsible for upwards of 180,000 deaths worldwide annually, mostly in immunocompromised individuals such as AIDS patients. Currently there are no licensed fungal vaccines, and even with anti-fungal drug treatment, cryptococcal meningitis is often fatal. Thus, Cryptococcosis remains a significant cause of morbidity and mortality world-wide.

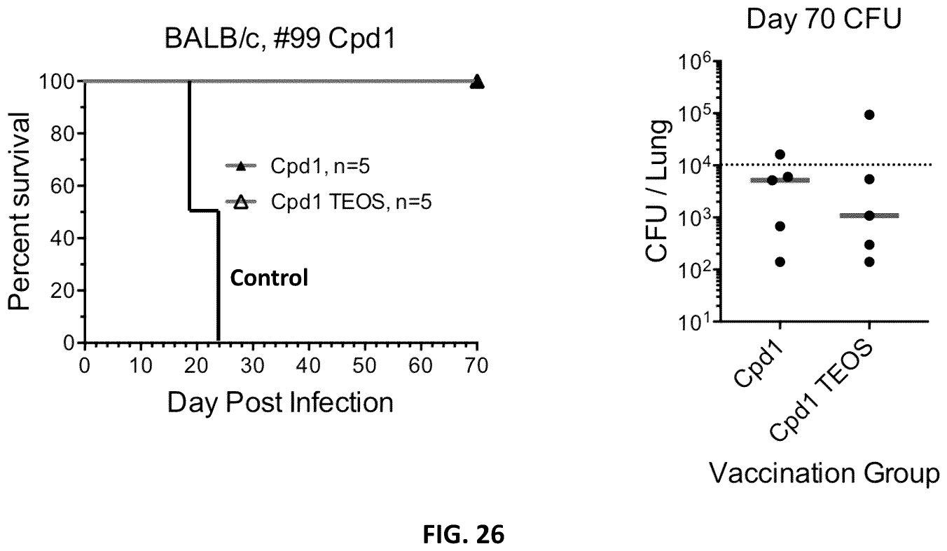

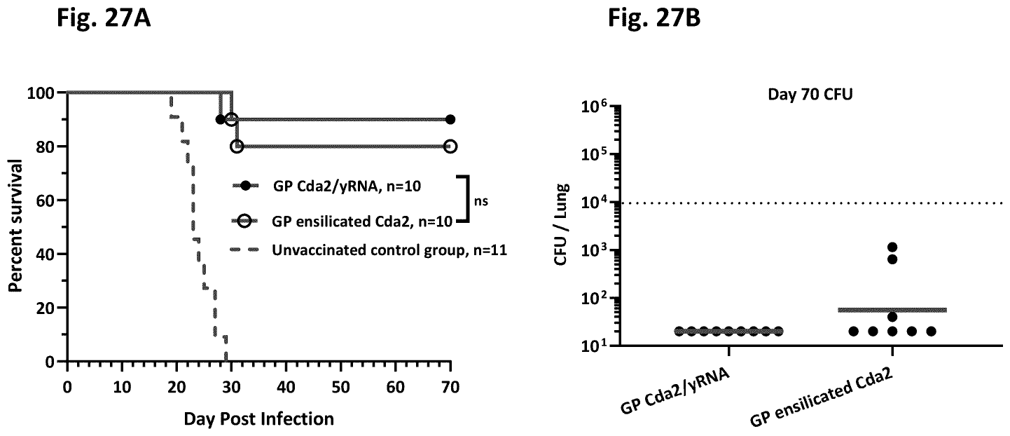

To develop and test the efficacy of cryptococcal vaccines in mice, vaccines were prepared by encapsulating cryptococcal protein antigens carboxy peptidase (Cpd1) or chitin deacetylase (Cda2) in GPs. Antigens were either trapped in GPs using yRNA (GP Cpd1 MSA/yRNA or GP Cda2 MSA/yRNA) or ensilicating antigen in GPs with TEOS (GP Cpd1 TEOS or GP Cda2 TEOS). Mice (BALB/c) were immunized with GPs carrying each single antigen payload. Vaccinated and unvaccinated mice were challenged with a lethal cryptococcal infection and percent survival was observed. FIG. 26A shows that almost all unvaccinated (control) mice died around 25 days post infection while 100% of the mice vaccinated with the Cpd1 antigen (either trapped with yRNA or ensilicated within GPs) survived for 70 days post infection. FIG. 26B shows that in the vaccinated mice that received GPs with ensilicated antigen showed fewer bacterial colony forming units (CFUs) in the lung that mice vaccinated with yRNA trapped antigen. This indicates that ensilicated antigen promoted a stronger protective immunity in vaccinated mice. FIG. 27A shows that mice vaccinated with the Cda2 antigen in GPs (either trapped with yRNA or ensilicated) had a better survival rate than mice the unvaccinated mice. The cryptococcal colony forming units (CFU) in each group of surviving mice was measured on day 70 post exposure. Vaccination with Cda2 antigen encapsulated in GPs with yRNA or ensilication both reduced the cryptococcal CFUs significantly as seen in FIG. 27B.

### Example 12: Ensilication of Lysozyme (Lys, MW=14 kD)

Ensilication Efficiency with Lysozyme Payload

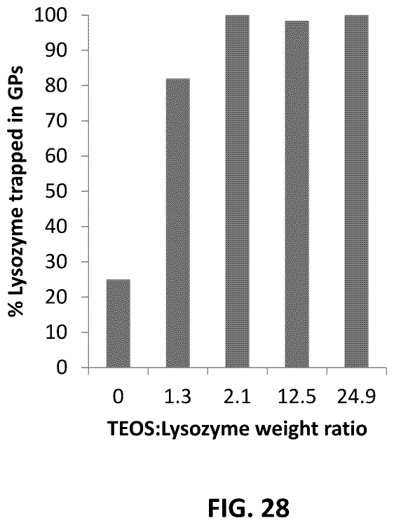

GPs were loaded with lysozyme (Lys) using the same procedure described above for loading of BSA in GPs. TEOS was prepolymerized at pH 2 and the lysozyme payload inside GPs was ensilicated as described before. FIG. 28 shows the efficiency at which lysozyme was ensilicated in GPs at various TEOS:lysozyme w/w ratios.

Thermal Stability of Ensilicated Lysozyme

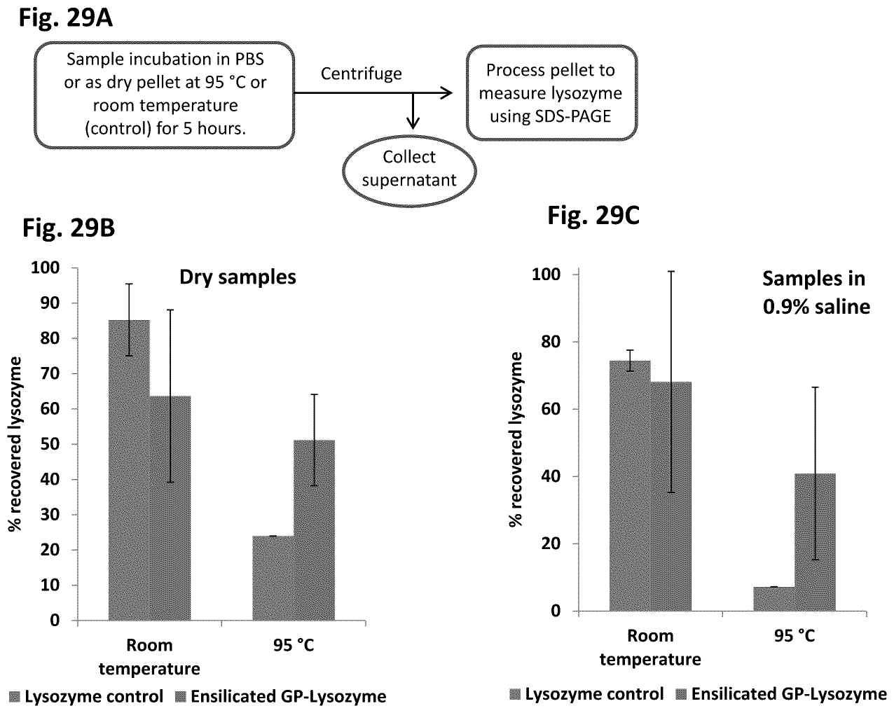

Lysozyme was loaded in GPs by the procedure as described above for BSA. The lysozyme payload was then ensilicated using TEOS (TEOS:Lysozyme ratio 12.5:1) as described before for BSA. GPs containing ensilicated lysozyme were incubated in dry form or in 0.9% saline at 95° C. or room temperature for 5 hours. Samples were centrifuged and supernatants discarded. The BSA in the pellets was extracted with 6M urea and SDS loading dye. The extracted BSA was centrifuged. The precipitated protein in the pellets was discarded and BSA remaining in the supernatant was quantified by SDS-PAGE. FIG. 29A summarizes the protocol used to evaluate the percent of lysozyme remaining in GPs after prolonged incubation at given temperatures. FIG. 29 shows percent of lysozyme remaining in dry (FIG. 29B) or 0.9% saline+/−heating (FIG. 29C) GP-lysozyme samples after prolonged exposure to room or high temperature. Free BSA and BSA encapsulated in GPs by trapping with yRNA were used as control.

Activity of Ensilicated Lysozyme after Exposure to High Temperature

The activity of the ensilicated lysozyme recovered from GPs after incubation at 95° C. for 5 hours in the experiment above was also tested. Lysozyme activity was quantified by spectrophotometric measurement of p-nitrophenol (λ_{abs}=405 nm) released following reaction of lysozyme with p-nitrophenyl β-D-N,N′,N″-triacetyl-chitotriose. The lysozyme assay used is described by Osawa et al. (Toshiaki Osawa, Yasuo Nakazawa, Lysozyme substrates. Chemical synthesis of p-nitrophenyl O-(2-acetamido-2-deoxy-(3-D-glucopyranosyl)-(1→4)-2-acetamido-2-deoxy-β-D-glucopyranoside and its reaction with lysozyme, Biochim. Biophys. Acta, 130 (1966) 56-63.)

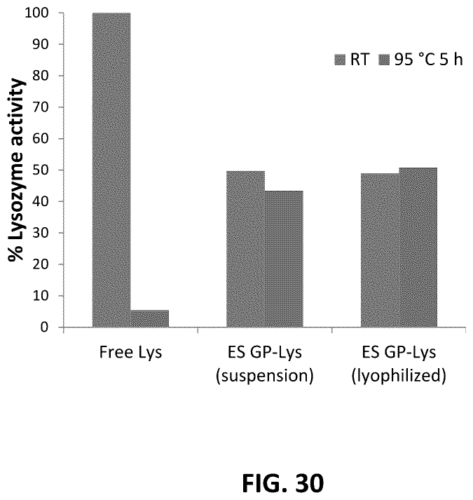

FIG. 30 shows the percent lysozyme activity retained after exposure to high temperature in samples containing free lysozyme or GP with ensilicated lysozyme (either dry/lyophilized sample or sample suspended in 0.9% saline). Results show that ensilication protects lysozyme activity during prolonged high temperature incubation.

Short Term Storage Stability of GP-Ensilicated Lysozyme

The storage stability of free lysozyme, lysozyme encapsulated within GPs, lysozyme trapped in GPs with yRNA, and lysozyme ensilicated within GPs was tested after incubation at 45° C. for a total of 144 hours. Samples were stored either as dry, lyophilized powders or liquid samples stored in PBS with 2 mM sodium azide (NaN₃). Lysozyme activity was assayed as described above before incubation and after 24 hour period.

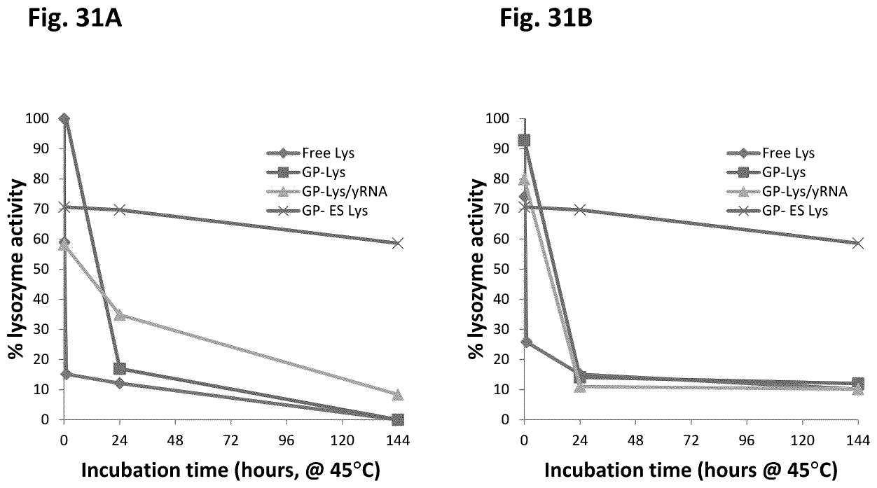

FIG. 31 shows the percent lysozyme activity at various time points during incubation. Free lysozyme (Lys) and GP-Lys samples lost>85% bioactivity within 1-24 h incubation at 45° C. Lysozyme trapped in GPs using yRNA as trapping agent lost>85% bioactivity after 24 hours (sample stored in PBS suspension) or 7 days (dry powder) of incubation at 45° C. GP Ensilicated (ES) lysozyme incubated at 45° C. retained >50% bioactivity after incubation at 45° C. for >7 days. The half-life (T½) for thermal inactivation of free lysozyme was <1 hour and for GP ES LYS was >144 hours.

Long Term Storage Stability of GP-Ensilicated Lysozyme

The storage stability of free lysozyme and lysozyme ensilicated within GPs was tested after incubation at 45° C. for a total of 90 days. Samples were stored either as dry, lyophilized powders or liquid samples stored in PBS with 2 mM sodium azide (NaN₃). Lysozyme activity was assayed as described before incubation and after 15 days.

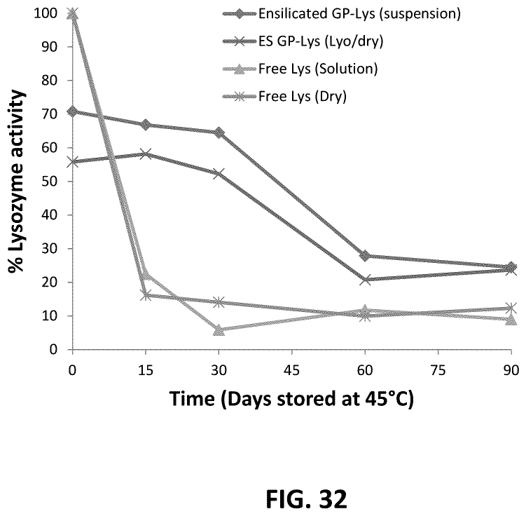

FIG. 32 shows the percent lysozyme activity at various time points during incubation. GP ensilicated lysozyme incubated at 45° C. retained >50% bioactivity for >30 days.

### Example 13: Ensilication of Other Payloads

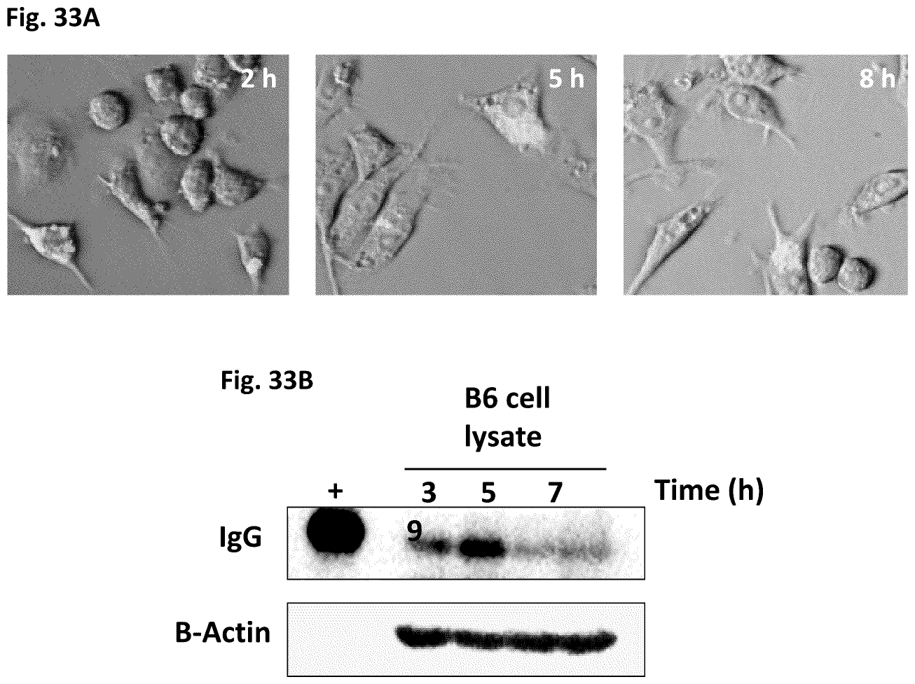

Ensilication of IgG antibody (MW=150 kD) Antibodies (IgG 488) were loaded and ensilicated within GPs by the same process described before for BSA. GPs loaded with ensilicated IgG (IgG 488-(SiO₂)ₙ) were allowed to be phagocytosed by cells of the B6 cell line. FIG. 33A shows that IgG was released inside macrophage cells and diffused throughout the cytoplasm. FIG. 33B shows that intact IgG was detected in B6 cells incubated with GP-IgG-(SiO₂)ₙ.

Ensilication of Peptides

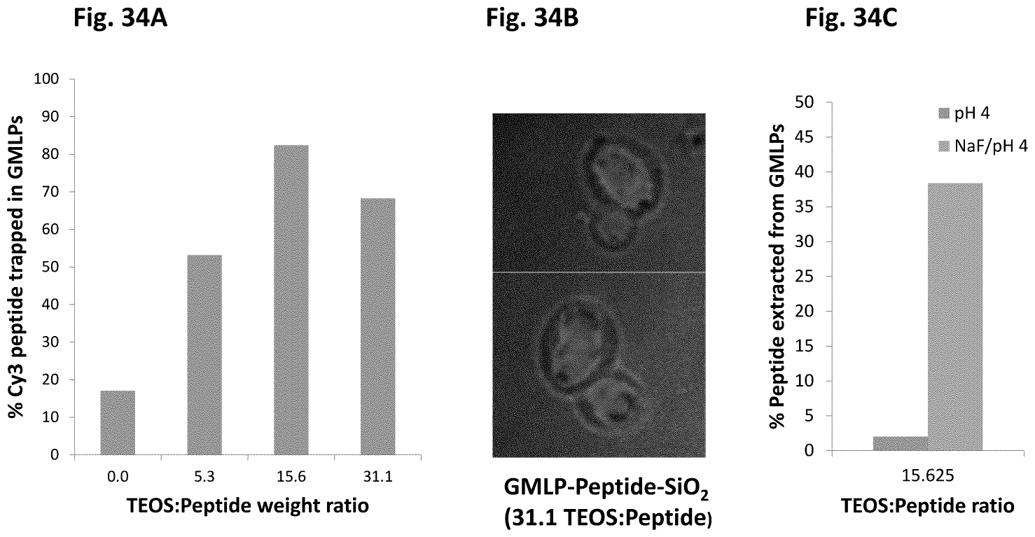

Hairpin peptide (MW 4570.96) and Cda2 Peptide 1 were loaded and ensilicated within GPs or glucan mannan lipid particles (GMLPs) by the same process described before for BSA. Percent of fluorescently labeled hairpin peptide ensilicated within GMLPs was assessed at various TEOS:peptide weight ratios. Over 80% of hairpin peptide was ensilicated within GMLPs when the TEOS:peptide ratio was 15.6 (FIG. 34A). Fluorescent micrograph showed that the peptide was trapped within GMLPs (FIG. 34B). Hairpin peptide ensilicated within GMLPs using a TEOS:peptide ratio of 15.6 was extracted from GMLPs at pH 4 using hydrochloric acid (HCl) or sodium fluoride (NaF). Extraction was most efficient when NaF was used (FIG. 34C).

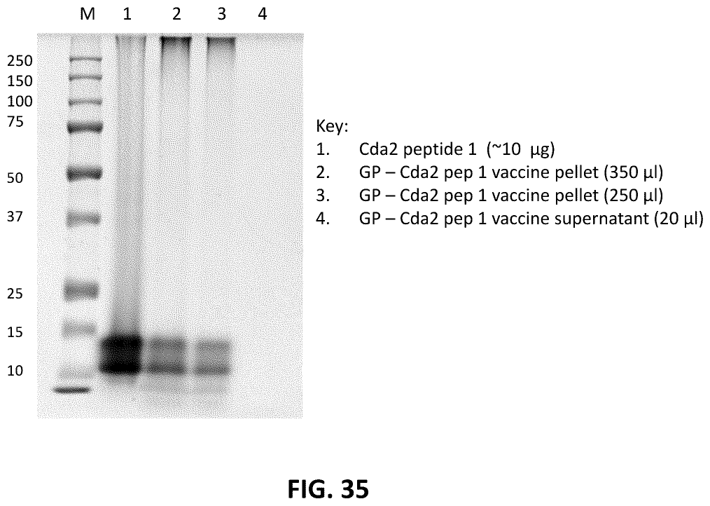

Cda2 peptide 1 was also ensilicated efficiently in GPs as it was detected in the GP pellet but not in the supernatant (FIG. 35).

Ensilication of Nucleic Acids

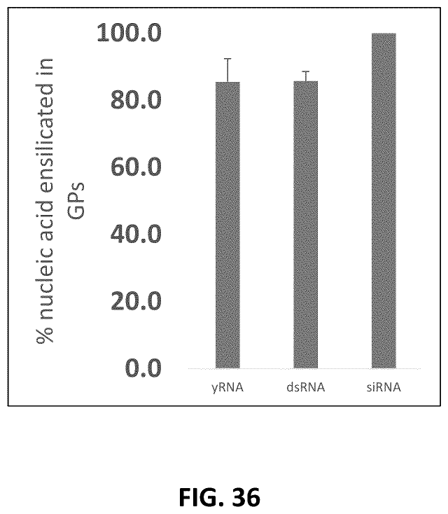

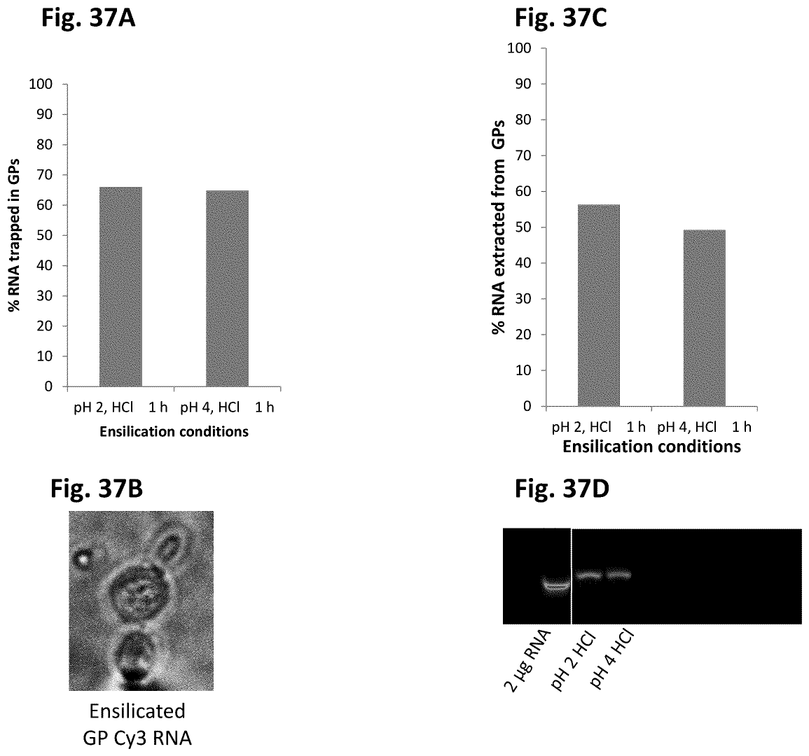

Different types of RNAs, yRNA, dsRNA and siRNA, were loaded and ensilicated within GPs by the same process described above for BSA. Ensilication was carried out using 10% 3-aminopropyltriethoxysilane (APTES) and 90% tetraethylorthosilicate (TEOS). in the presence of pH 2 or pH 4 HCl. After ensilication, intact RNA was extracted from GPs using 200 mM NaF at pH 4. FIG. 36 shows different RNAs could be encapsulated within GPs at over 80% efficiency. Encapsulation was equally efficient when Cy3 RNA and 100% TEOS in HCl at pH 2 and 4 (FIG. 37A). Fluorescent micrographs showed that RNA was located inside GPs (FIG. 37B). RNA ensilicated in HCl at pH 2 or pH 4 was efficiently extracted with NaF (FIGS. 37C and 37D).

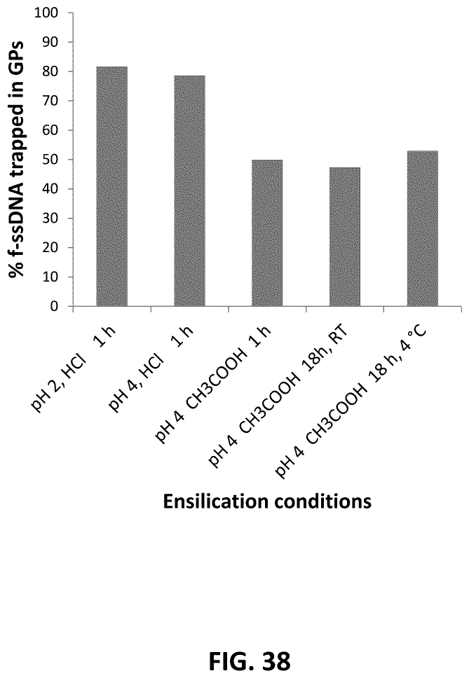

FITC labeled salmon sperm DNS (ssDNA) was also efficiently ensilicated within GPs when TEOS was prepolymerized at pH 4 in the presence of HCl or acetic acid (CH3COOH). TEOS prepolymerized in acetic acid was incubated with DNA payload containing GPs at room temperature (RT) or 4° C. for 1 hour or 18 hours (FIG. 38).

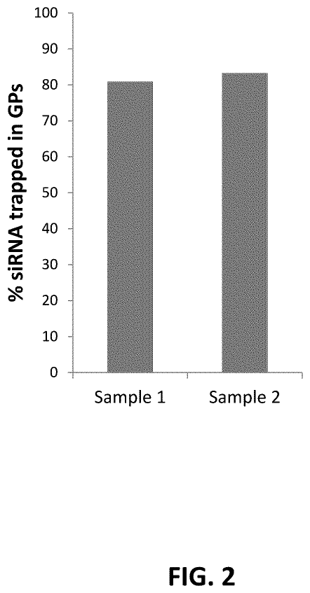

Similarly, Cy3-siRNA was also efficiently ensilicated within GPs using TEOS prepolymerized at pH 2 using HCl (FIG. 2).

Ensilication of Proteins with 100% TEOS

To test whether proteins with different physical properties could be encapsulated in GPs, lysozyme, ovalbumin, transferrin, or glucose oxidase were loaded in GPs using 100% TEOS. The physical properties of the proteins are listed in Table 2.

TABLE 2
Physical properties and encapsulation
efficiency of different proteins.

| | Molecular Weight | Isoelectric Point | % encapsulation |
|---|---|---|---|
| Protein | (kD) | (pI) | efficiency in GPs |
| Lysozyme | 14.5 | 11.1 | 90 ± 8 |
| (LYS) | | | |
| Ovalbumin | 45 | 4.5 | 97 ± 3 |
| (OVA) | | | |
| Transferrin | 80 | 6.3-6.4 | 92 ± 8 |
| (TFN) | | | |
| Glucose | 160 (dimer) | 4.2 | 98 ± 3 |
| oxidase | 80 (monomer) | | |
| (GOx)* | | | |

*GOx is loaded and encapsulated as dimer (160k). It is extracted and quantified as monomer by SDS-PAGE as a protein denaturing solution is necessary for quantitative extraction.

Dry GPs were mixed with a solution of 25 mg/mL protein in water (4 μL protein solution per mg GP, 100 μg protein per mg GP), incubated to allow for complete absorption of protein solution inside the hollow cavity of GPs, and then the samples were frozen and lyophilized. To maximize protein incorporation into GPs, the dry GP-OVA samples were hydrated with 2 μL water per mg GP, mixed, frozen, and lyophilized. TEOS (Millipore Sigma, Burlington, MA, USA; #86578, ≥99% purity, density 0.933 g/mL) was mixed with a 0.01 M HCl (pH 2) solution at a volume ratio of 2:1 (v/v) aqueous HCl:TEOS and the sample was incubated at 20° C. with constant mixing for one hour to allow for partial polymerization of TEOS. Then, the partially polymerized single phase TEOS solution was added to dry GP-protein (4 μL per mg GP) and the sample was incubated for one hour. GP ensilicated protein samples were suspended in 0.9% sterile saline (5 mg GP/mL), centrifuged and the supernatants were collected to quantify unencapsulated protein. The samples were then washed three more times in 0.9% sterile saline, suspended in 0.9% saline at 5 mg GP/mL and stored at 20° C.

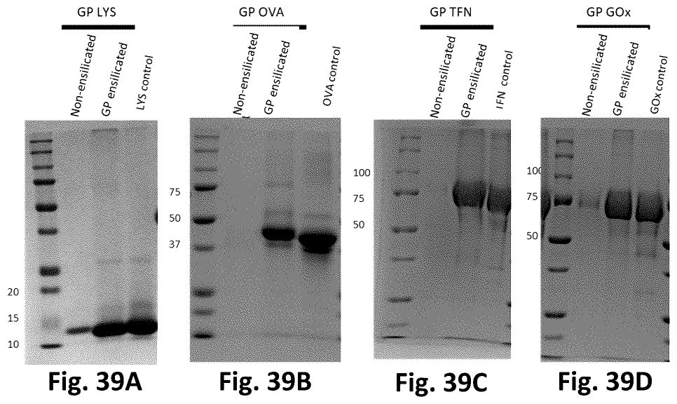

GP ensilicated protein was extracted and quantified by following the procedure described above for BSA. Proteins in the GP supernatants and pellets were quantified by SDS-PAGE. FIG. 39 shows proteins were predominantly present in the pellet indicating that protein was encapsulated at a high efficiency regardless of their molecular weight and isoelectric points. Quantification of proteins in the pellets indicated that the proteins were encapsulated at a greater than 90% encapsulation efficiency (Table 2).

### Example 14: Transfection Using mRNA Encapsulated in GPs

The transfection efficiency of mRNA encapsulated in GPs via ensilication was tested. 3T3-D1 cells were transfected with the control GPs containing encapsulated yRNA:25k PEI: mCherry mRNA along with an endosomal release excipient and the test sample GPs containing ensilicated mCherry mRNA without an endosomal release excipient. Ensilicated samples were prepared with optimized method (10% APTES+90% TEOS) for ensilication of nucleic acids as described above.

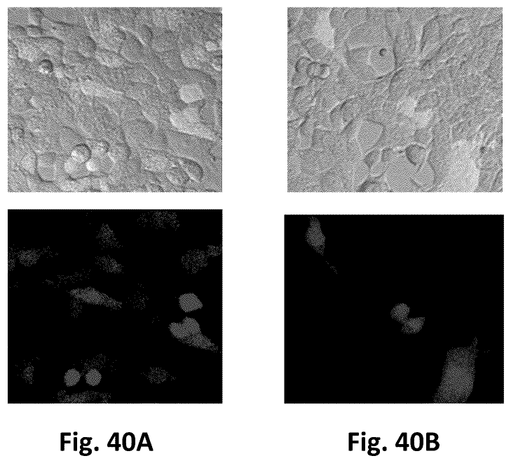

3T3-D1 cells transfected by GPs were viewed using brightfield and fluorescence microcopy to detect the expression of the mCherry protein. FIG. 40 shows fluorescence micrographs with and without an overlay of a brightfield micrograph showing mCherry protein fluorescence in 3T3-D1 cells following transfection with GP yRNA:25k PEI: mCherry mRNA control (FIG. 40A) and GP ensilicated mCherry mRNA (FIG. 40B).

The results indicate that mRNA ensilicated in GPs was effectively transfected into cells and translated.

### LITERATURE CITED

Toshiaki Osawa, Yasuo Nakazawa, Lysozyme substrates. Chemical synthesis of p-nitrophenyl O-(2-acetamido-2-deoxy-β-D-glucopyranosyl)-(1→4)-2-acetamido-2-deoxy-β-D-glucopyranoside and its reaction with lysozyme, Biochimica et Biophysica Acta (BBA)—General Subjects, Volume 130, Issue 1, 1966, Pages 56-63, ISSN 0304-4165, https://doi.org/10.1016/0304-4165(66)90008-0.
Buckley, A. M., & Greenblatt, M. (1994). The sol-gel preparation of silica gels. Journal of Chemical Education, 71(7), 599-602.
Chen, Y C., Smith, T., Hicks, R. et al. Thermal stability, storage and release of proteins with tailored fit in silica. Sci Rep 7, 46568 (2017). https://doi.org/10.1038/srep46568.
Wahid, A. A., Doekhie, A., Sartbaeva, A. et al. Ensilication Improves the Thermal Stability of the Tuberculosis Antigen Ag85b and an Sbi-Ag85b Vaccine Conjugate. Sci Rep 9, 11409 (2019). https://doi.org/10.1038/s41598-019-47657-9.
Aouadi M, Tesz G J, Nicoloro S M, Wang M, Chouinard M, Soto E, Ostroff G R, Czech M P. Orally delivered siRNA targeting macrophage Map4k4 suppresses systemic inflammation. Nature. 2009 Apr. 30; 458(7242):1180-4. doi: 10.1038/nature07774. PMID: 19407801; PMCID: PMC2879154.
Soto, E; Ostroff, G; Oral Macrophage Mediated siRNA Delivery System. Nanotech 2008 Volume 2, Chapter 4—Drug and Gene Delivery Systems, pages 332-335.
Ernesto R. Soto and Gary R. Ostroff. Characterization of Multilayered Nanoparticles Encapsulated in Yeast Cell Wall Particles for DNA Delivery Bioconjugate Chemistry 2008 19 (4), 840-848 DOI: 10.1021/bc700329p
Soto, E. R; Ostroff, G. R; Yeast Cell Wall Particles As A Versatile Macromolecular Delivery System. PMSE Preprints (2008), 98 591-593.
SOTO, E; OSTROFF, G. R; Oral Macrophage Mediated Gene Delivery System. Nanotech 2007 Volume 2, Chapter 5—Drug Delivery, pages 378-381.
Soto, E; Ostroff, G; Glucan Particles as Carriers of Nanoparticles for Macrophage-Targeted Delivery, Nanomaterials for Biomedicine, Chapter 3, 2012, pp 57-79, ACS Symposium Series, Volume 1119.
Soto E R, Caras A C, Kut L C, Castle M K, Ostroff G R. Glucan particles for macrophage targeted delivery of nanoparticles. J Drug Deliv. 2012; 2012:143524. doi: 10.1155/2012/143524. Epub 2011 Oct. 13. PMID: 22013535; PMCID: PMC3195348.
Soto, E; Rus, F; Ostroff, G. Yeast Cell Wall Particle Encapsulation of Pro-Terpene Payloads, 2019, TechConnect Briefs 2019, 435-438
Calo, J. R.; Crandall, P. G.; O'Bryan, C. A.; Ricke S. C. Essential Oils as Antimicrobials in Food Systems—A Review. Food Control. 4602015, 54, 111-119.
Bakry, A. M.; Abbas, S.; Ali, B.; Majeed, H.; Abouelwafa, M. Y.; Mousa, A.; Liang, L. Microencapsulation of Oils: A Comprehen-462sive Review of Benefits, Techniques, and Applications. Compr. Rev. Food Sci. Food Saf. 2016, 15, 143-182. 463.
Bhalerao, Y. P.; Wagh S. J. A Review on Thymol Encapsulation and its Controlled Release through Biodegradable Polymer Shells. 465Int. J. Pharm. Sci. 2018, 2, 4522-4532. doi: 10.13040/IIPSR.0975-8232.9(11).4522-32 466.
Gómez, B.; Barba, F. J.; Domínguez, R.; Putnik, P.; Bursać Kovac̆ević, D.; Pateiro, M.; Toldrá, F.; Lorenzo, J. M. Microencapsula-467tion of Antioxidant Compounds through Innovative Technologies and Its Specific Application in Meat Processing. Trends Food 468Sci. Technol. 2018, 82, 135-147.
Saifullah, M.; Shishir, M.; Ferdowsi, R.; Rahman, M.; Van Vuong, Q. Micro and Nano Encapsulation, Retention and Controlled Release of Flavor and Aroma Compounds: A Critical Review. Trends Food Sci. Technol. 2019, 86, 230-251. 471.
Wagner, T. E, Vaccine Delivery Systems Using Yeast Cell Wall Particles, 2017, U.S. Pat. No. 10,166,195, and United States Patent Application publication 2017/0007688 A1

1. A nano-silica yeast particle (YP) delivery system comprising a YP with a hollow inner cavity and at least one first payload,
wherein the at least one first payload is substantially or completely encapsulated by a nano-silica cage, and
wherein the nano-silica cage and the at least one first payload are both substantially encapsulated within the hollow inner cavity of the YP.

2. (canceled)

3. The nano-silica in YP delivery system of claim 1, wherein the YP is selected from the group consisting of a yeast cell wall particle (YCWP), a glucan particle (GP), a yeast glucan particle (YGP), a yeast glucan-mannan particle (YGMP), a glucan lipid particle (GLP), a whole glucan particle (WGP), a glucan mannan lipid particle (GMLP), and a glucan chitin particle (GCP), or any mixtures thereof.

4. The nano-silica YP delivery system of claim 1, wherein the at least one first payload is selected from the group consisting of a protein, a peptide, a peptide antigen, an enzyme, an antibody, a nanobody, an antigen binding fragment of an antibody, a single stranded nucleic acid, and a double stranded nucleic acid, or any mixtures thereof.

5. The nano-silica YP delivery system of claim 1, wherein the nano-silica cage comprises a chemical selected from the group consisting of tetraethylorthosilicate (TEOS), tetraethylorthogermanate (TEOG), tetramethylorthosilicate (TMOS), aminopropyl triethoxysilicate (APTES), Bis[3-(triethoxysilyl)propyl]disulfide (BTEPDS), and 3-(triethoxysylil)-propyl-isocyanate (TEPI) or any combinations thereof.

6-7. (canceled)

8. The nano-silica YP delivery system of claim 1, further comprising a coating polymer in the hollow inner cavity,
wherein the coating polymer is located on the outside of the nano-silica cage or on the outside of the YP, and
wherein the coating polymer is nontoxic and has no pharmacologic activity,
wherein the coating polymer optionally resists breakdown in the presence of gastric fluids in the oral cavity, esophagus, or stomach or optionally disintegrates in the small intestine.

9-10. (canceled)

11. The nano-silica YP delivery system of claim 8, where in the polymer is chosen from the group consisting of methacrylic acid methylmethacrylate copolymer, methacrylic acid ethyl acrylate copolymer, cellulose acetate phthalate (CAP), cellulose acetate trimellate (CAT), hydroxy propyl methyl cellulose phthalate (HPMCP), dydroxyl propyl methyl cellulose acetate succinate (HPMCAS), polyvinyl acetate (PVAP), methacrylic acid polymer, and any combination thereof.

12. The nano-silica YP delivery system of claim 1, wherein the payload is stable after a short-term or a long-term exposure to high temperature, and wherein the temperature is optionally 25° C., 45° C., or 95° C.

13. (canceled)

14. The nano-silica YP delivery system of claim 12, wherein the payload is stable after exposure to the said high temperature for about 30 minutes, about 2 hours, about 5 hours, 15 days, 30 days, 45 days, 60 days, 75 days or 90 days.

15. The nano-silica YP delivery system of claim 1 further comprising one more additional payloads,
optionally wherein the one or more additional payloads is not confined in the nano-silica cage, or
optionally wherein the one or more additional payload is selected from the group consisting of a protein, a peptide, a peptide antigen, an enzyme, an antibody, an antigen binding fragment of an antibody, a single stranded nucleic acid, a double stranded nucleic acid, and a mixture thereof.

16. (canceled)

17. The nano-silica YP delivery system of claim 1 and a pharmaceutically acceptable carrier or excipient.

18. A kit comprising the nano-silica YP delivery system of claim 1 and optional instructions for use.

19. A method of preparing a nano-silica yeast particle (YP) delivery system comprising the steps of:
(a) loading a YP comprising a hollow inner cavity with at least one first payload; and
(b) resuspending the YP in prepolymerized tetrahydroorthosilicate (TEOS) in half hydrodynamic volume,
wherein the prepolymerized TEOS is prepolymerized at a pH of about 2 to about 4, wherein the TEOS polymerizes to form a nano-silica cage within the hollow inner cavity, and
wherein the nano-silica cage substantially or completely encapsulates the at least one first payload at an encapsulation efficiency of at least 90%, and
wherein the nano-silica cage and the at least one first payload are both confined within the hollow inner cavity of the YP.

20. (canceled)

21. The method of claim 19, further comprising the step of loading one or more additional payloads in the YP.

22. The method of claim 19, further comprising the step of loading a coating polymer in the YP.

23. A pharmaceutical composition comprising a nano-silica yeast particle (YP) delivery system comprising a YP with a hollow inner cavity and at least one first payload,
wherein the at least one first payload is substantially or completely encapsulated by a nano-silica cage, and
wherein the nano-silica cage and the at least one first payload are both confined within the hollow inner cavity of the YP.

24. (canceled)

25. The pharmaceutical composition of claim 23, wherein the at least one first payload is selected from the group consisting of a protein, a peptide, a nucleic acid, and any combination thereof.

26. A method of treating a disease condition in a subject, comprising administering the pharmaceutical composition of claim 23 to a subject in need thereof.

27. A vaccine comprising a nano-silica yeast particle (YP) delivery system comprising a YP with a hollow inner cavity and at least one first payload,
wherein the at least one first payload is substantially or completely encapsulated by a nano-silica cage, and
wherein the nano-silica cage and the at least one first payload are both confined within the hollow inner cavity of the YP.

28. (canceled)

29. The vaccine of claim 27, wherein the at least one first payload is selected from the group consisting of a protein, a peptide, a glycoprotein, a lipoprotein, a toxoid, a polysaccharide, and a nucleic acid or any combinations thereof.

30. A method of preventing a disease condition in a subject, comprising administering to the subject the vaccine of claim 27.
